(19) United States
(12) Patent Application Publication
(10) Pub. No.: US 2024/0327877 A1
(43) Pub. Date: Oct. 3, 2024

(54) Conversion of lignin to muconic acid and methods therefor

(71) Applicants: Alliance for Sustainable Energy, LLC, Golden, CO (US); Massachusetts Institute of Technology, Cambridge, MA (US); Wisconsin Alumni Research Foundation, Madison, WI (US); UT-Battelle, LLC, Oak Ridge, TN (US)

(72) Inventors: Gregg Tyler BECKHAM, Golden, CO (US); Chad Thomas PALUMBO, Boulder, CO (US); Allison Jean ZIMONT WERNER, Denver, CO (US); Kevin P. SULLIVAN, Golden, CO (US); Yuriy ROMÁN, Cambridge, MA (US); Shannon Scot STAHL, Madison, WI (US); Adam Michael GUSS, Knoxville, TN (US); Jay D. HUENEMANN, Knoxville, TN (US); Thomas David MAND, Oak Ridge, TN (US); Kathryn MAINS, Golden, CO (US); Austin Lee CARROLL, Knoxville, IN (US)

(21) Appl. No.: 18/625,942

(22) Filed: Apr. 3, 2024

Related U.S. Application Data
(60) Provisional application No. 63/463,584, filed on May 3, 2023.
(60) Provisional application No. 63/493,900, filed on Apr. 3, 2023.

(51) Int. Cl. C12P 7/18; C12N 1/20; C12R 1/40

(52) U.S. Cl. CPC C12P 7/18; C12N 1/205; C12P 2201/00; C12R 2001/40

(57) ABSTRACT

Described herein is a chemical process for the conversion of lignocellulosic biomass into muconic acid which is useful for the generation of plastics and polymers. The described methods utilize catalytic chemical reactions and biological processes to facilitate the conversion, while increasing yields and reducing energy requirements.

### CROSS-REFERENCE TO RELATED APPLICATIONS

This application claims priority from U.S. Provisional Patent Application Nos. 63/493,900, and 63/463,584 filed on Apr. 3, 2023 and May 5, 2023, respectively, the contents of which are incorporated herein by reference in their entirety.

### CONTRACTUAL ORIGIN

This invention was made with government support under Contract No. DE-AC36-08GO28308 awarded by the Department of Energy. The government has certain rights in the invention.

The United States Government has rights in this invention pursuant to contract no. DE-AC05-000R22725 between the United States Department of Energy and UT-Battelle, LLC.

### BACKGROUND

Muconic acid is an important precursor material for the production of consumer plastics, including nylon, polyurethane and polyethylene terephthalate. Lignin is the largest source of renewable aromatics available in nature comprising between 15-30% of lignocellulosic biomass. More than 90 million dry-tons of lignin are harvested annually. Accordingly, the conversion of lignin to muconic acid provides an opportunity for the production of consumer plastics with a reduced reliance on petrochemicals and an expected reduction in green house gas emissions.

### SUMMARY

Described herein is a chemical process for the conversion of lignocellulosic biomass into muconic acid which is useful for the generation of plastics and polymers. The described methods utilize catalytic chemical reactions and biological processes to facilitate the conversion, while increasing yields and reducing energy requirements.

The described reduction catalytic fractionation and hydrodeoxygenation for the processing of lignin are described in U.S. Provisional Patent Application 63/395,067 filed Aug. 4, 2022, which is hereby incorporated by reference in its entirety.

In an aspect, provided is a method comprising: a) providing a lignocellulosic biomass reactant; b) fractionating the lignocellulosic biomass reactant via reductive catalytic fractionation (RCF), thereby generating a RCF oil; c) deoxygenating the RCF oil via hydrodeoxygenation (HDO), thereby generating a HDO oil; d) oxidizing the HDO oil, thereby generating a plurality of oxygenated monomers; and e) bioconverting the plurality of oxygenated monomers in the presence of a bacterium, thereby generating muconic acid. The lignocellulosic biomass reactant may comprise lignin.

The step of fractionating may be performed in the presence of a RCF catalyst, for example, Mo₂C. The step of fractionating may have a greater than 75%, 80%, 90%, 95%, or optionally 99% conversion of lignocellulosic biomass reactant to RCF oil.

The step of deoxygenating may be performed in the presence of an HDO catalyst, for example, Mo₂C. The step of deoxygenating may have a greater than 75%, 80%, 90%, 95%, or optionally 99% conversion of RCF oil to HDO oil.

The step of oxidizing may be performed in the presence of one or more oxidation catalysts, for example, Co(OAc)₂, Mn(OAc)₂, Zr acetylacetonate and NaBr. Each of the oxidation catalysts may be provided at a weight percentage selected from the range of 0.5% to 10%, 0.5% to 5%, or 1% to 20%. The step of oxygenating may have a greater than 50%, 60%, 70%, 80%, or optionally 85% conversion of HDO oil oxygenated monomers. The oxidation catalyst may comprise Co(OAc)₂, Mn(OAc)₂ and NaBr at a ratio of 5:5:1, respectively.

The oxygenated monomers may comprise, for example, benzoic acid, phthalic acid, terephthalic acid, isophthalic acid, hemimellitic acid, benzene tricarboxylic acid, biphenyl dicarboxylic acid or a combination thereof.

The step of bioconverting may be performed in the presence of a genetically engineered bacterium, for example, a genetically modified strain of Psuedomonas putida.

In an aspect, provided is a genetically modified bacterium comprising a genetically modified strain of Psuedomonas putida KT2440, wherein the Psuedomonas putida KT2440 is capable of converting benzoate and terephthalate into muconate. The bacterium may have the modification fpva:Ptac:tpaKRHA1 where fpva:Ptac:tpaKRHA1 enables terephthalate uptake and tpaK is a heterologously expressed gene from Rhodococcus jostii RHA1. The bacterium may have the modification ΔhsdMR::Ptac:tphA2II: tphA3II:tphBII:tphA1IIE6, where ΔhsdMR::Ptac:tphA2II:tphA3II:tphBII:tphA1IIE6 enables terephthalate conversion to protocatechuate and tphA2II, tphA3II, tphBII, and tphA1II are heterologously expressed genes from Comamonas sp. E6. The bacterium may have the modification Ptac:aroYEc:ecdBEc:ecdDEc, where Ptac:aroYEc:ecdBEc:ecdDEc enables protocatechuate conversion to catechol and aroY, ecdB, and ecdD are heterologously expressed genes from Enterobacter cloacae. The bacterium may have the modification ΔpcaHG, where ΔpcaHG prevents catabolism of protocatechuate. The bacterium may have the modification ΔcatRBC::Ptac:catA, where ΔcatRBC::Ptac:catA enables catechol conversion to muconate and prevents catabolism of muconate.

The bacterium may have the modification ΔampC::P_{tac}:ophC:ophK Δcrc::P_{tac}:ophA2:ophA1:ophB:ophP enabling ortho-phthalate uptake and conversion to protocatechuate. The bacterium may have the deletion of pcaD (ΔpcaD) to prevent the catabolism of muconolactone.

### BRIEF DESCRIPTION OF DRAWINGS

Some embodiments are illustrated in referenced figures of the drawings. It is intended that the embodiments and figures disclosed herein are to be considered illustrative rather than limiting.

FIG. 1 provides an example scheme and representative molecules of the multistep process for the conversion of lignocellulosic biomass to muconic acid.

FIG. 2 provides high-performance liquid chromatography (HPLC) data of the HDO oil and the oxygenation monomers, illustrating the efficiency of the oxidation step as described herein.

FIG. 3 illustrates the bioconversion of the oxygenated monomers as described herein, over time in the presence of the bacterium. The concentration of muconic acid (MA) is increased significantly.

FIG. 4 illustrates the mSAGE cycle overview. Initial introduction of poly-attB landing pads via allelic exchange follow by multi-plexed integration of up to nine unique DNA elements.

FIG. 5A illustrates growth enrichment workflow. Twenty total plasmids were pooled in equal mass amounts and electroporated into AG5577. Following transformation, the population was enriched with antibiotic selection and then pre-adapted to minimal media with an aromatic carbon source. The population was then divided among three shakes flask and 48-well plate wells containing minimal media with IPA as the sole carbon source. Populations were enriched over four rounds of subculturing top identified optimal genetic combinations. FIG. 5B provides representative growth data for plate reader and shake flasks depicting changes in growth rates over time. FIG. 5C shows population sequencing to show dynamic change in detectable genetic targets.

FIG. 6A provides individual serine recombinase integrating attP plasmids were co transformed with recombinase expression plasmid. FIG. 6B provides colony formation was evaluated to quantify transformation efficiency. Twenty-four isolates were screened via PCR for verification of integration accuracy at expected locus.

FIG. 7A is a schematic of multi-plexed DNA integration. Six total plasmids including three integrating attP plasmids and three recombinase expression plasmids were pooled and transformed into the poly-attB landing pad strain AG5577. FIG. 7B provides colony formation on selective media containing kanamycin (50 μg/ml), gentamicin (30 μg/ml), and Spectinomycin/Streptinomycin (200/300 μg/ml).

FIG. 8 is a visual depiction of simultaneous plasmid backbone excision via transient expression of fC31 recombinase. pGW31 is transformed into strains with integrated attP plasmids to catalyze the recombination of attP and attB sites to remove plasmid backbones containing the origin of replication and antibiotic resistance markers. This results in a minimal attL site on the chromosome and the formation of a non-replicating plasmid which is lost through cell division over time.

FIG. 9 illustrates the catabolic steps to convert IPA to PCA. Plasmid maps depicting mSAGE plasmids with IPA orthologs.

FIG. 10 provides a Sankey showing the mass balance of poplar biomass as described herein.

FIG. 11 shows the effect of the reaction parameters on the outcome of autoxidation reaction of HDO oil under milder conditions.

FIG. 12 provides an illustration of the total monomer content of HDO oil.

FIG. 13 provides a gas chromatogram showing the monomers produced by hydrodeoxygenation of poplar reductive catalytic fractionation lignin oil. The monomers were identified by electron impact mass spectroscopy.

FIG. 14 shows a comparison of GC chromatograms of HDO autoxidation catalysis (after methyl derivatization) using a 5 wt % Co(OAc)₂·4H₂O/5 wt % Mn(OAc)₂·4H₂O/5 wt % Zr(acac)₄/10 wt % NHPI at 200° C. with 6 bar 02 over 18 h (top—blue) and a 5 wt % Co(OAc)₂·4H₂O/5 wt % Mn(OAc)₂·4H₂O/5 wt % Zr(acac)₄/5 wt % NaBr. catalyst at 200° C. with 6 bar O₂ over 3 h. Retention times are slightly shifted due to changes in the column in the duration between data collection of the two reactions.

FIG. 15 illustrates the carbon balance of multiple passes as described herein.

FIGS. 16A-16E provide catabolic pathways for TPA, IPA and OPA in KT2440 enables growth (AW521) or muconolactone production (KMM001) from oxidized HDO lignin (HDOx) monomers. (FIG. 16A) Engineered P. putida KT2440 strains that metabolize terephthalic acid (TPA), isophthalic acid (IPA), phthalic acid (OPA) and benzoic acid (BA) for growth (AW521) or muconolactone production (KMM001). (FIG. 16B) All four HDOx monomers are utilized by AW521 as the sole carbon source for growth in shake flasks. OD600 for each culture is displayed as open hexagons in the color corresponding to the carbon source. (FIG. 16C) AW521 consumes all four monomers when grown on 10 mM total aromatics (2.5 mM each monomer) in shake flasks. (FIG. 16D) KMM001 utilizes glucose (fed to 20 mM every 24 hours) for growth, and converts BA, TPA, and IPA to muconolactone in shake flasks when 2.5 mM of each HDOx monomer is present. The strain does not consume OPA. (FIG. 16E) KMM001 converts BA, TPA, and IPA to muconolactone at nearly 100% yields in both cultures (shake flasks) with individual monomers and in a “mix” case (as seen in 16C) but does not convert OPA.

FIGS. 17A-17B show the growth of engineered P. putida KT2440 strains on 10 mM phthalic acid in M9 minimal media (30 mL shake flasks; 30° C., 225 rpm). (FIG. 17A) Cellular growth of (OD600) and (FIG. 17B) phthalate consumption by each strain tested. KMM026 grows the fastest on 10 mM OPA.

### DETAILED DESCRIPTION

The embodiments described herein should not necessarily be construed as limited to addressing any of the particular problems or deficiencies discussed herein. References in the specification to “one embodiment”, “an embodiment”, “an example embodiment”, “some embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used herein the term “substantially” is used to indicate that exact values are not necessarily attainable. By way of example, one of ordinary skill in the art will understand that in some chemical reactions 100% conversion of a reactant is possible, yet unlikely. Most of a reactant may be converted to a product and conversion of the reactant may asymptotically approach 100% conversion. So, although from a practical perspective 100% of the reactant is converted, from a technical perspective, a small and sometimes difficult to define amount remains. For this example of a chemical reactant, that amount may be relatively easily defined by the detection limits of the instrument used to test for it. However, in many cases, this amount may not be easily defined, hence the use of the term “substantially”. In some embodiments of the present invention, the term “substantially” is defined as approaching a specific numeric value or target to within 20%, 15%, 10%, 5%, or within 1% of the value or target. In further embodiments of the present invention, the term “substantially” is defined as approaching a specific numeric value or target to within 1%, 0.9%, 0.8%, 0.7%, 0.6%, 0.5%, 0.4%, 0.3%, 0.2%, or 0.1% of the value or target.

As used herein, the term “about” is used to indicate that exact values are not necessarily attainable. Therefore, the term “about” is used to indicate this uncertainty limit. In some embodiments of the present invention, the term “about” is used to indicate an uncertainty limit of less than or equal to ±20%, ±15%, ±10%, ±5%, or ±1% of a specific numeric value or target. In some embodiments of the present invention, the term “about” is used to indicate an uncertainty limit of less than or equal to ±1%, ±0.9%, ±0.8%, ±0.7%, ±0.6%, ±0.5%, ±0.4%, ±0.3%, ±0.2%, or ±0.1% of a specific numeric value or target.

The provided discussion and examples have been presented for purposes of illustration and description. The foregoing is not intended to limit the aspects, embodiments, or configurations to the form or forms disclosed herein. In the foregoing Detailed Description for example, various features of the aspects, embodiments, or configurations are grouped together in one or more embodiments, configurations, or aspects for the purpose of streamlining the disclosure. The features of the aspects, embodiments, or configurations, may be combined in alternate aspects, embodiments, or configurations other than those discussed above. This method of disclosure is not to be interpreted as reflecting an intention that the aspects, embodiments, or configurations require more features than are expressly recited in each claim. Rather, as the following claims reflect, inventive aspects lie in less than all features of a single foregoing disclosed embodiment, configuration, or aspect. While certain aspects of conventional technology have been discussed to facilitate disclosure of some embodiments of the present invention, the Applicants in no way disclaim these technical aspects, and it is contemplated that the claimed invention may encompass one or more of the conventional technical aspects discussed herein. Thus, the following claims are hereby incorporated into this Detailed Description, with each claim standing on its own as a separate aspect, embodiment, or configuration.

### Example 1—Conversion of Lignin to Muconic Acid

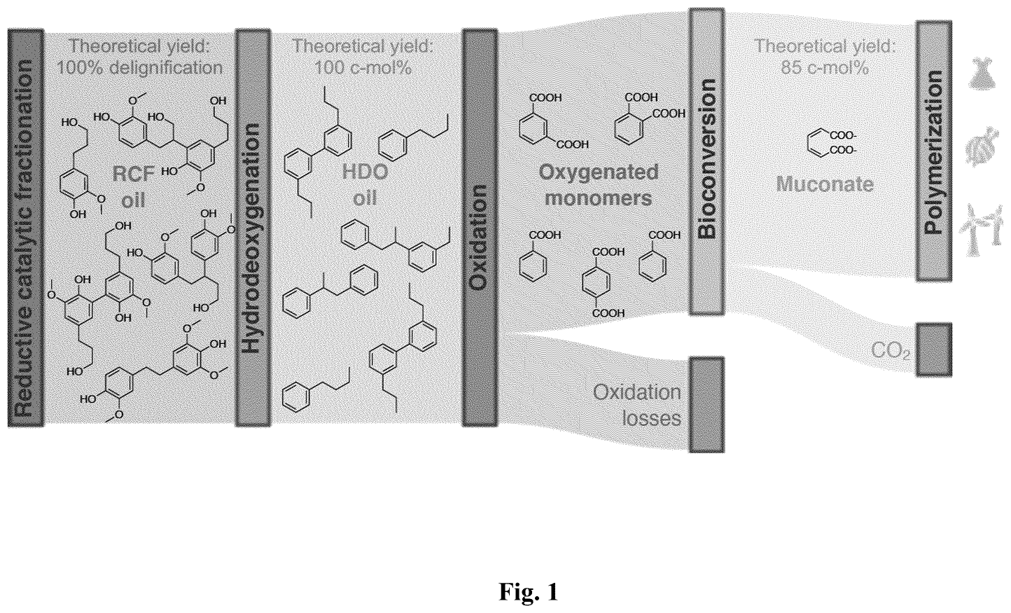

The present example illustrates the conversion of lignin in biomass to muconate through a combination of chemical and biological processes (FIG. 1). First, three sequential chemical deconstruction steps are applied: reductive catalytic fractionation (RCF), hydrodeoxygenation (HDO), and autoxidation, generating a mixture of oxygenated monomers. Value is derived from the mixture of oxygenated monomers by a bioconversion step. To do this, a novel engineered non-natural strain of Pseudomonas putida was engineered to convert benzoate and terephthalate to muconate.

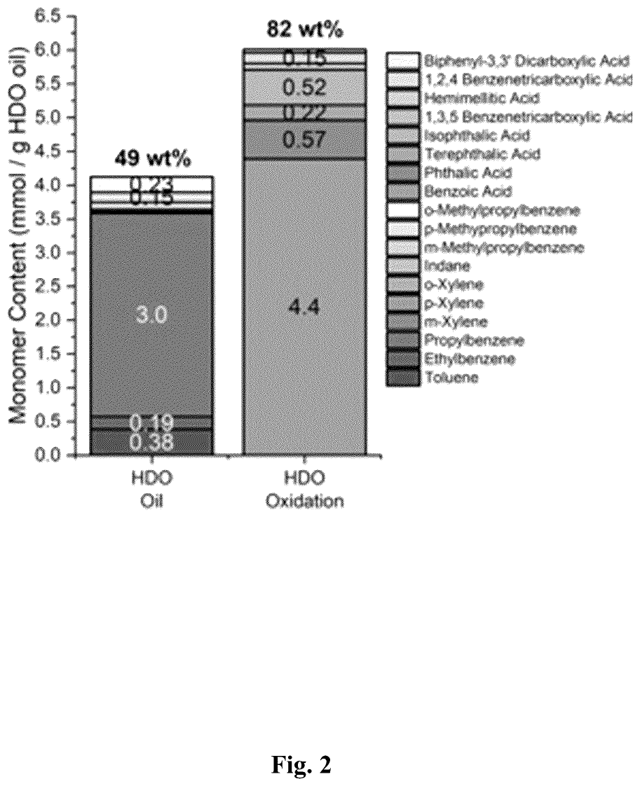

The generation of mixed monomer oxygenates was accomplished in corrosion resistant titanium batch reactors equipped with glass liner inserts purchased from the Parr Instrument Company. For a single reaction, the vessel was charged with HDO oil (50-500 mg), acetic acid (15 mL), a stir bar, along with the catalyst comprising 0.5-10 wt % each of Co(OAc)₂·4H2O, Mn(OAc)₂·4H2O, Zr(acac)4 (acac=acetylacetonate), and NaBr. The mixture was purged and then charged with zero air and nitrogen to achieve 1-8 bar O₂ at 10% of the total loading pressure. The vessel was heated to 180-220° C. and held for 1-3 h before it was cooled rapidly in an ice bath. The solutions were analyzed by HPLC (FIG. 2), and a total monomer yield of 82 wt % was quantified. The 6.01 mmol monomer/g of HDO oil yield represents a 146 mol % increase in monomer content with respect to the 4.13 mmol monomer/g HDO oil value of the HDO substrate. The increase in moles of monomer is attributed to C—C bond cleavage of lignin oil oligomers.

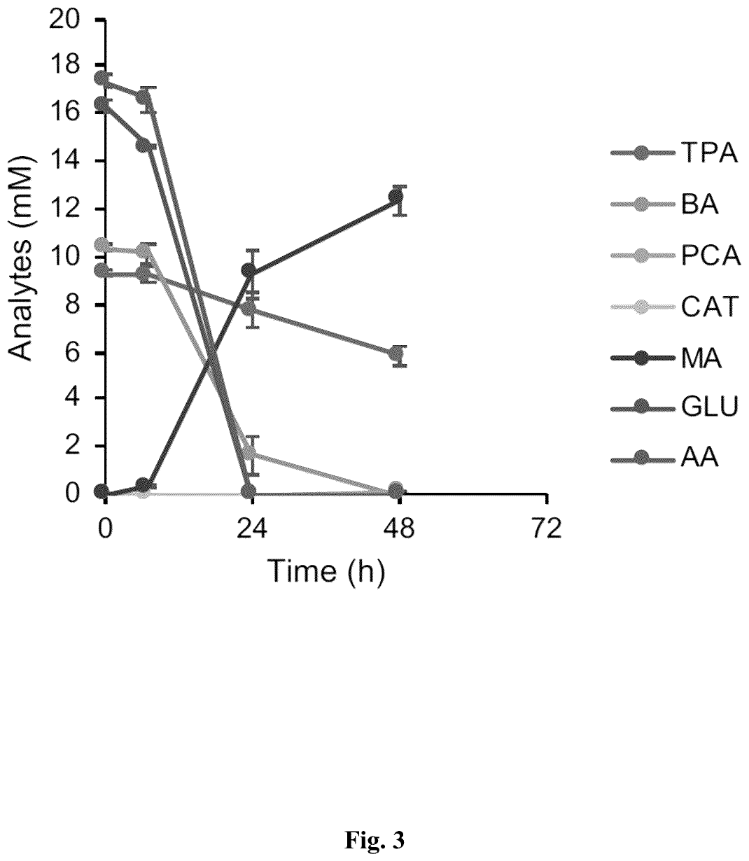

FIG. 3 shows the bioconversion of the oxygenated monomers in the presence of a genetically engineered strain of Psuedomonas putida. Bioconversion of a mixture of benzoate and terephthalate to muconate by AW415 was demonstrated in shake flasks. AW415 was cultivated in M9 minimal medium (pH 7.2, 6.78 g/L Na2HPO4, 3 g/L KH2PO4, 0.5 g/L NaCl, 1 g/L NH4Cl, 2 mM MgSO4, 100 μM CaCl2), and 18 μM FeSO4) supplemented with 10 mM benzoate, 10 mM terephthalate, 20 mM glucose, and 20 mM acetate. Muconate was produced at a titer of 12.4±0.6 mM, yield of 0.63±0.02 mol/mol, and carbon balance of 93±1%.

The genetically modified Psuedomonas putida, as described herein, was made using chromosomal deletions or insertions performed by homologous recombination.

### Example 2—Catabolism of Isophthalic Acid

### Background

Genetic engineering of microbes is a promising route to develop biotechnological solutions for many challenges facing society today, such as human healthcare, resilient agriculture, environmental pollution, and sustainable chemical production. Many microbes have evolved functions that would help address these challenges. However, most microbes are not optimized to perform any specific tasks beyond those necessary for growth and survival. Natural microbial phenotypes are complex and can be easily disrupted by both biotic and abiotic factors; as such, optimization of these phenotypes often requires complex engineering solutions to alter gene expression, native metabolic flux, and native regulatory systems. Further, developing bioprocesses with these organisms can introduce perturbations to native metabolic stasis that effects substrate turnover or production generation. Synthetic biology has made it possible to engineer microbes capable of addressing these challenges. Yet, advanced synthetic biology tools, such as those for efficiently performing multiple chromosomal modifications, are limited to highly developed model organisms, rather than the non-model organisms that harbor the attributes required for many biotechnological applications.

Targeted chromosomal engineering is critical for developing robust microbial biotechnologies that function in challenging environments, but most existing tools for integrating DNA into chromosomes have significant drawbacks. For example, many function in limited ranges of organisms, insert DNA at random locations, require replicating plasmids, leave selection markers in the host, require multiple steps for selection marker excision, or are limited to just one or two DNA insertions.

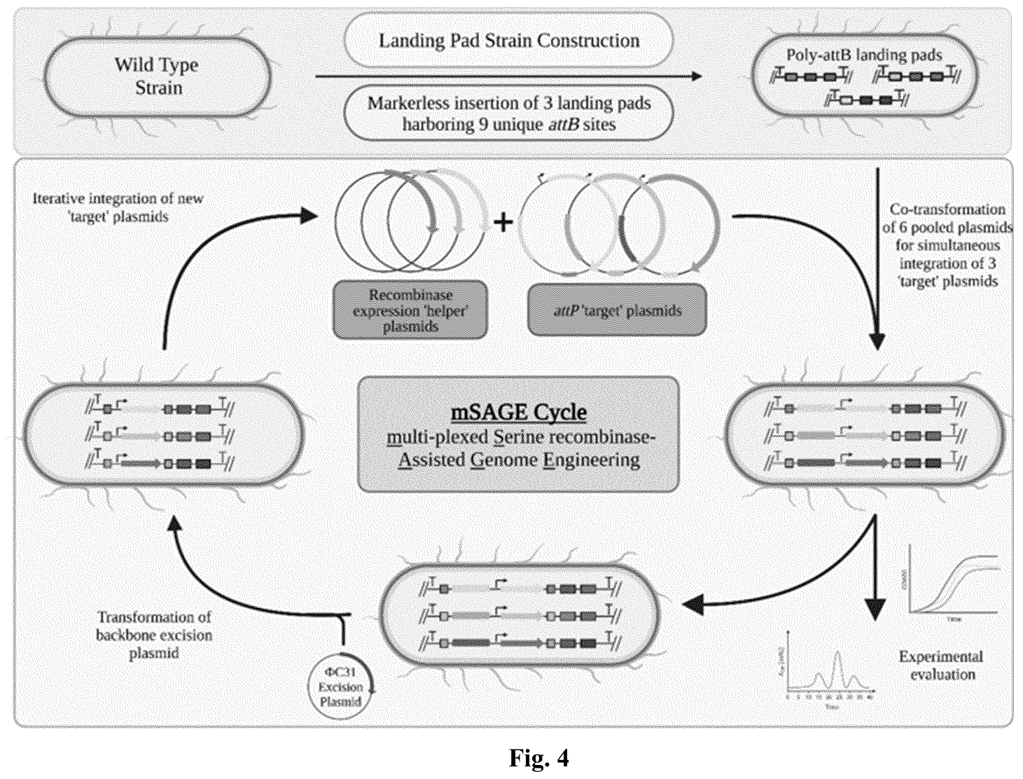

Serine recombinase-Assisted Genome Engineering (SAGE) is an efficient organism-agnostic chromosomal engineering tool that allows 9 cycles of highly efficient DNA insertion into the host chromosome, thus addressing these limitations. Here, we introduce multiplexed SAGE (mSAGE), which extends the SAGE toolkit to enable simultaneous chromosomal integration of three heterologous DNA molecules and recycling of three antibiotic markers with a single excision step (FIG. 4). Strain construction with SAGE on par with the fastest genome engineering methods, and multiplexing SAGE further accelerates the strain construction process by 4-fold.

Pseudomonas putida KT2440 has emerged as a promising host for many industrial applications due to its robust redox capacity, solvent tolerances, metabolic regime and has been shown to withstand industrial stress conditions. This exceptional potential was most recently highlighted by its use in a tandem chemical-biological process for simultaneous recycling of mixed plastic waste. In recent years, significant strides have been made to more efficiently engineer the P. putida chromosome. Nonetheless, these tools suffer from the same drawbacks that led to the development of SAGE, and similar to many other organisms the low throughput of genetic engineering hampers the use of P. putida as an industrial host chassis. Thus, we chose P. putida as a platform organism for both prototyping the mSAGE toolkit.

The original SAGE toolkit is very modular, with easily swappable genetic parts, and we exploit this modularity to both enable multiplex integration and greatly improve the SAGE toolkit. The cargo delivered by each attP plasmid is insulated by multiple (synthetic and natural) terminator sequences to reduce transcriptional interference between neighboring operons. Nonetheless, as increasingly more highly expressed genetic programs are installed at a given chromosomal locus, the potential for interference increases. Accordingly, we split the original poly-attB cassette into three landing pads, each insulated by rho-independent terminators, and integrated each landing pad into three distal chromosomal loci to generate the mSAGE base strain AG5577. This split has the beneficial side effect of further enhancing the chromosomal stability of highly modified strains by making recombination between shared genetic elements lethal.

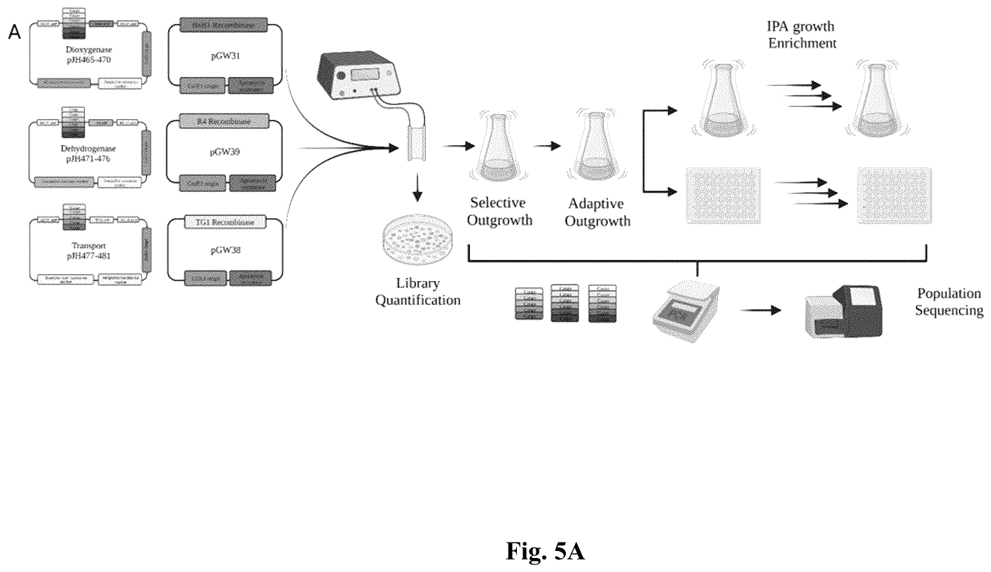

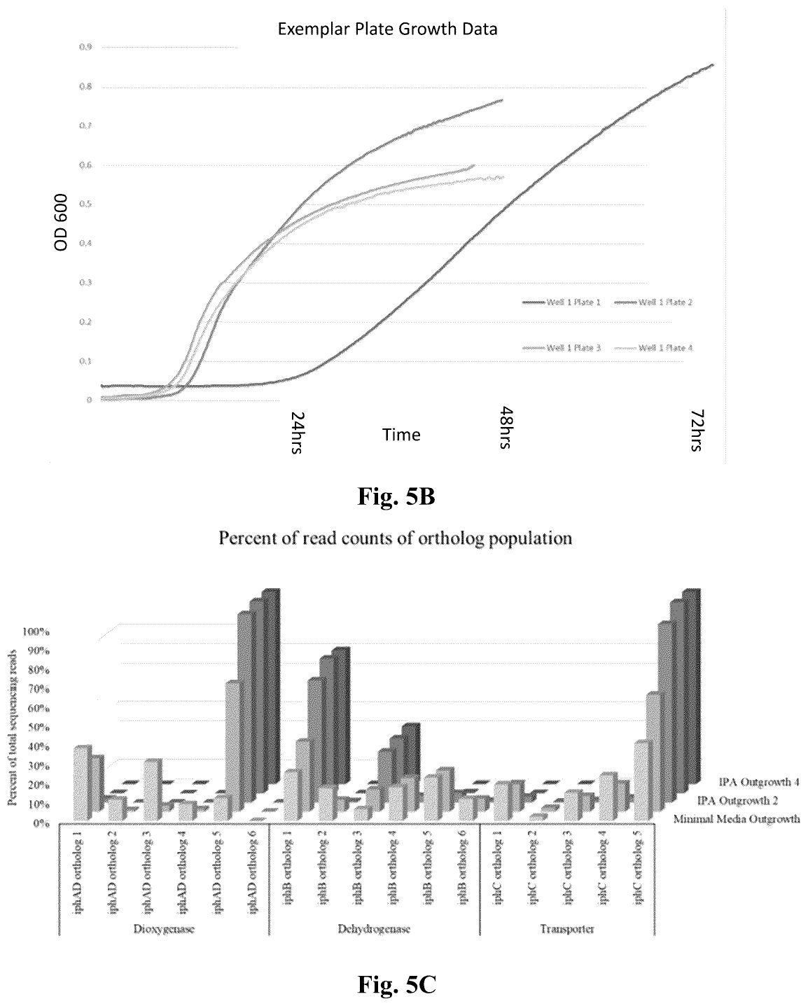

By generating a new generation of attP target plasmids we simultaneously enable multiplex plasmid integration, simplify cloning cargo that is toxic in E. coli, and allow use of SAGE in kanamycin insensitive bacteria. For this, we replaced the nptII(kanamycin resistance) and the pUC origin of replication from the original pJH204-212 attP target plasmids with the following: (1) aac(3)-IIa (gentamicin resistance) and medium copy ColA origin, (2) aadA (spectinomycin resistance) and low copy CloDF13 origin (FIGS. 5A-5B). An additional variant of all three target plasmid families was generated that retained the pUC origin, and included the bla (ampicillin resistance) selection marker in addition to the existing nptII, aacC(3), or aadA marker to simply third party plasmid synthesis (Supplementary FIG.). Toxicity of heterologous gene expression, which often leads to plasmid mutation, can be eliminated through use of attP target plasmids with the ColA or CloDF13 origins. Additionally, recombination potential is greatly reduced when attP target plasmids must be co-integrated at the same chromosomal loci.

P. putida KT2440 engineered to use terephthalic acid and ethylene glycol can be used to convert a portion of mixed plastic waste in a hybrid chemical-biological process, but introduction of heterologous pathways for catabolism of other components of deconstructed plastic is essential for such processes to be carbon efficient and commercially viable.

### DESCRIPTION

Disclosed herein are methods and compositions of matter that enable an organism, such as bacteria, to catabolize isopthalic acid (IPA). In one embodiment, the bacteria is P. putida.

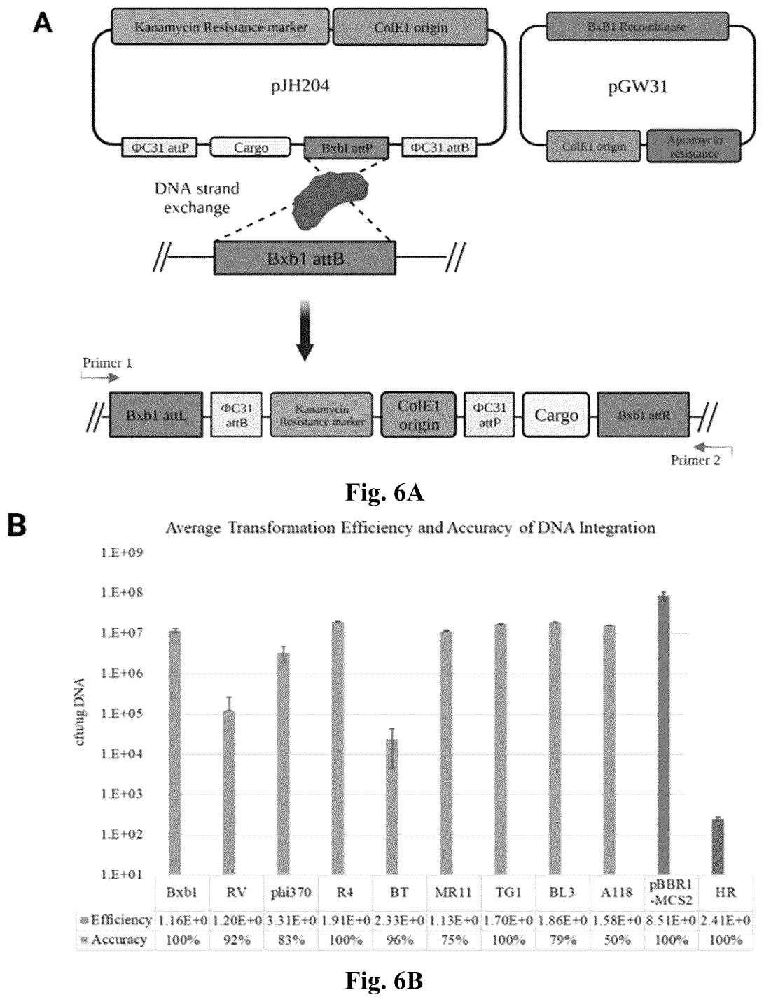

mSAGE was used to rapidly optimize a heterologous pathway that allows P. putida to catabolize isopthalic acid (IPA), a non-native carbon source that is significant component of PET plastics. A pathway for IPA catabolism in Comamonas sp. E6 has been previously described (FIGS. 6A-6C). Enzyme ortholog activity varies substantially in heterologous hosts and evaluation of different combinations of orthologs is essential to optimize catabolism. To identify orthologs for the Comamonas pathway, we isolated IPA-consuming bacteria from the environment and performed in silico analyses of their genomes to identify orthologs of the iphAD (dioxygenase), iphD (dehydrogenase) and iphC (permease) genes (Table 1). Expression cassettes for six orthologs of iphAD, six orthologs of iphB, and five orthologs of iphC were designed to simultaneously integrate into separate landing pads at the Bxb1, R4, and TG1 attB sites, respectively.

A growth selection was utilized to identify ortholog combinations that enable the most rapid growth on IPA in P. putida. We developed a workflow to enrich and adapt the population to allow for active growth selection with IPA as the sole carbon source (FIGS. 6A-6C). From a pooled transformation, a library of ˜32,000 transformants was generated, providing ˜175× coverage of the 180 possible gene ortholog combinations. Direct transfer from rich medium into defined medium can cause stochastic lag phases with some aromatic carbon sources. So, the pooled strain library was pre-adapted to assay conditions by cultivation in mineral medium with p-coumaric acid as the sole carbon source. Growth was observed after twenty-four hours and peaked within seventy-two hours, demonstrating the first transfer of IPA catabolism into a heterologous host/P. putida. The pooled library was sub-cultured in IPA for several additional passages to enrich for the fastest growing strains.

Samples were collected prior to and following each sub-cultures to track enrichment of ortholog variants. A pooled PCR reactions and subsequent Illumina amplicon sequencing were performed to the relative ratio of each ortholog in the population. The original population was diverse, but a purifying selection for a subset of orthologs occurred rapidly. Within two passages, iphAD and iphC were dominated by a single variant, and iphB stabilized with apparently equivalent variants. (FIGS. 6A-6C). To validate this methodology, we constructed ortholog combinations mirroring those identified by the competition assay. Numerous ortholog combinations enabled growth on IPA. However, the most rapid growth on IPA required the combination of orthologs that dominated the population in the competition assay. This demonstrates that our selective process successfully enriched the population to the optimal ortholog combinations to achieve robust utilization of IPA as a substrate. Establishing this catabolic pathway in P. putida expands the biological funnel required for carbon efficient plastic waste bioconversion and demonstrates the utility of mSAGE for generation of chromosomally-integrated combinatorial libraries for metabolic engineering.

mSAGE Base Strain and Plasmid Construction

Previously reported SAGE tools were modified and expanded to enable multi-plexed, simultaneous DNA integration into the chromosome of P. putida KT2440. We re-designed and constructed new plasmids to insert three attB sites with spacer sequences in between to allow for PCR screening to verify integration events. The landing pad sites were flanked with rho-independent terminators to reduce transcriptional read-through into and out of the site. Using plasmids pGW97-99 the re-designed landing pads were integrated into the chromosome of P. putida via allelic exchange to create the base mSAGE strain, AG5577. FIG. 6A depicts the poly-attB site organization and chromosomal insertion loci. Recombinase expression ‘helper’ plasmids (pGW31-33,35-40) were not altered from their original description as part of the SAGE toolkit. We expanded the functionality of the original integrating attP plasmids (pJH204-212) by constructing new plasmids with alternate resistance markers and origins of replication. The initial kanamycin resistance gene (nptII) was replaced by either a gentamicin resistance gene (AAC(3)-IIa, pJH213-221) or a spectinomycin resistance gene (aadA, pJH222-230). The ColE1 origin of replication was replaced with either the ColA (pJH213-221) or CloDF13 (pJH222-230) origin of replication to prevent insertion of identical DNA that could induce undesired recombination events within multi-integrated strains. To simplify external vendor DNA synthesis, alternate plasmids (pJH401-427) wherein the pJH204-230 origins of replication were all replaced with the ColE1 origin and a secondary resistance gene, bla, enabling ampicillin resistance in E. coli. General representation of the structural layout for all integrating attP plasmids are depicted in FIG. 6B. Together, this established a base strain and plasmid suite to evaluate and demonstrate mSAGE functionality as a tool for high efficiency DNA integration.

Transient Expression of Serine Recombinases Enables Efficient Unidirectional Integration of Single and Multiple Plasmid DNA Elements into Chromosome

A co-transformation assay of a single integrating attP plasmid and the appropriate recombinase expression plasmid was performed to evaluate the efficiency and accuracy of DNA integration. FIG. 5A depicts the organization and layout of DNA following co-transformation and chromosomal integration of the target plasmid. In triplicate, pJH204-212 were individually transformed with the non-replicating recombinase expression plasmid and plated on selective media. For comparison, a replicating plasmid (pBBR1-MCS2) and a homologous recombination plasmid (pGW97) were transformed to provide relative efficiency data for those methods. Following overnight growth, quantitative evaluation of the colonies formed for all transformations was completed. FIG. 5B shows the resulting average cfu/μg DNA for all attP plasmids as well as the replicating and homologous recombination plasmids. For all recombinases used, DNA integration efficiency significantly outperformed the homologous recombination plasmid by several orders of magnitude. Many of the recombinases enabled chromosomal DNA integration at nearly the same efficiency (1-3×10⁷ cfu/μg DNA) as the replicating plasmid control (8.5×10⁷ cfu/μg DNA).

Serine recombinases preferentially integrate DNA between their native att sites, but are known to potentially integrate into pseudo-att sites. Therefore, integration at the expected attB site was evaluated by a PCR screen of twenty-four isolates from each transformation. Shown in FIG. 3B, most recombinases were found to have exceptional accuracy of integration. Three recombinases, (BxbI, R4, TG1) were found to enable integration with 100% accuracy, five exceeded 70% (RV, ϕ370, BT, BL3, MR11), and only one (A118 integrase) was found to be less than 50% accurate. Together, these data demonstrate the high efficiency and accurate DNA integration capabilities of the mSAGE tools to provide unidirectional and stable genome editing tools in P. putida.

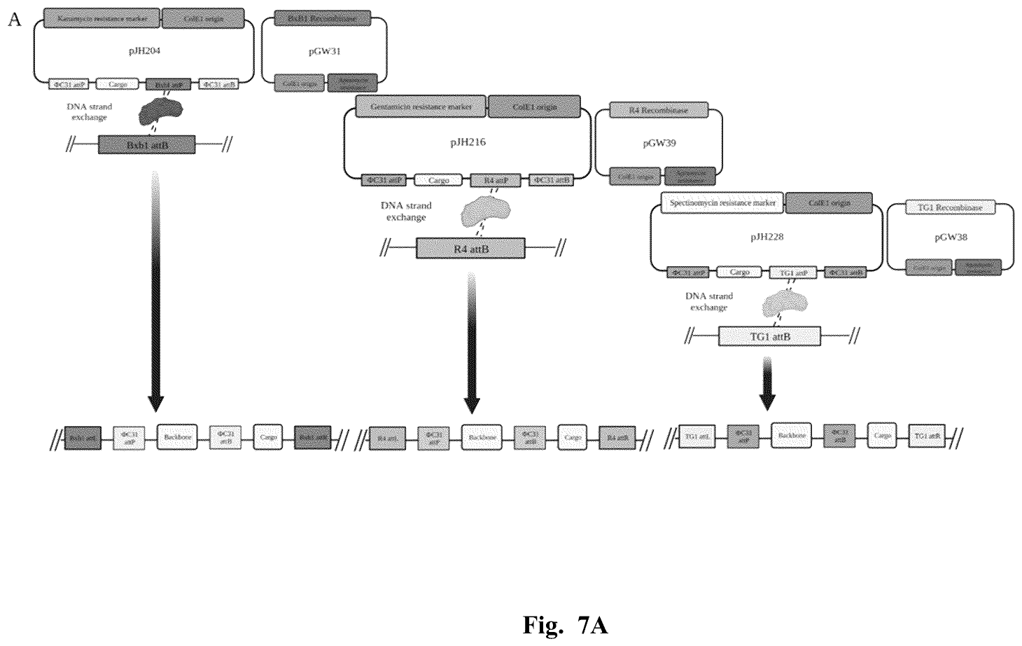

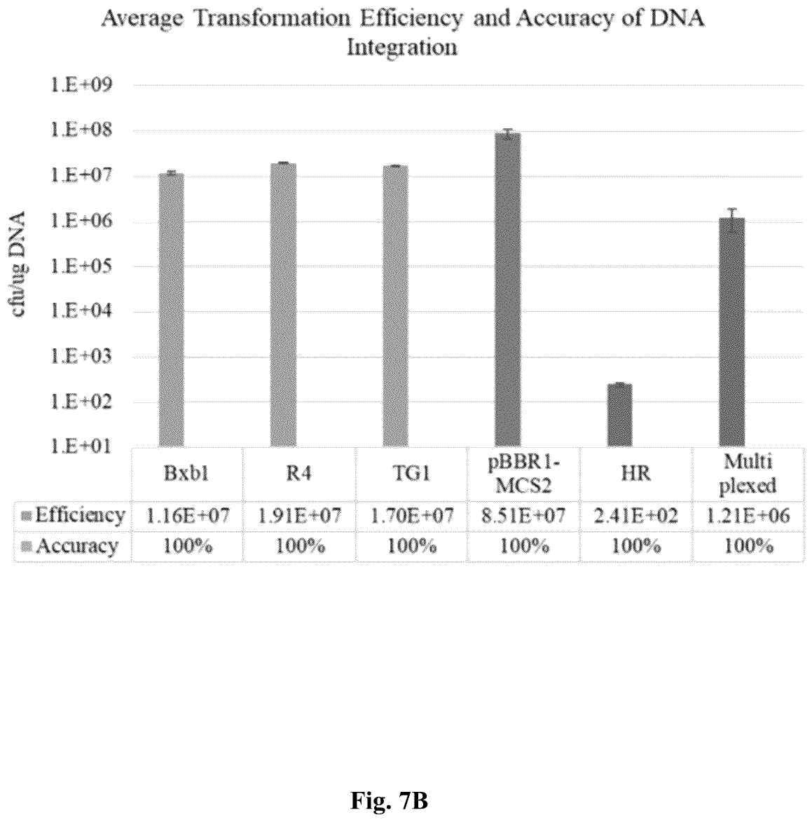

The observed high efficiencies of single recombinase DNA integration suggested that it could be possible to co-integrate multiple plasmids simultaneously. We assessed this by pooling equal mass (100 ng) amounts of three integrating attP plasmids (pJH204-216-228) along with their corresponding recombinase helper plasmids (pGW31-38-39), totaling 6 plasmids in the pool (FIG. 7A). This pool was transformed in the same manner as single recombinases and plated on selective media with all required antibiotics. Quantitative assessment of the colony formation was performed to determine the efficiency of simultaneous co-integration. FIG. 7B shows that co-integration of three plasmids simultaneously was possible and achieved levels of efficiency near that of single recombinases and the replicating plasmid. Further, integration accuracy was evaluated via PCR screening of twenty-four isolates. We verified integration for all three plasmids at the expected loci in 100% of the isolates screened. This data establishes mSAGE tools as the most efficient strategy for stable integration of DNA elements into the chromosome of P. putida and represents the first demonstration of simultaneous integration of three unique DNA elements.

Transient Expression of (Φ31 Recombinase Excises Plasmid Backbones and Permits Iterative mSAGE Cycles

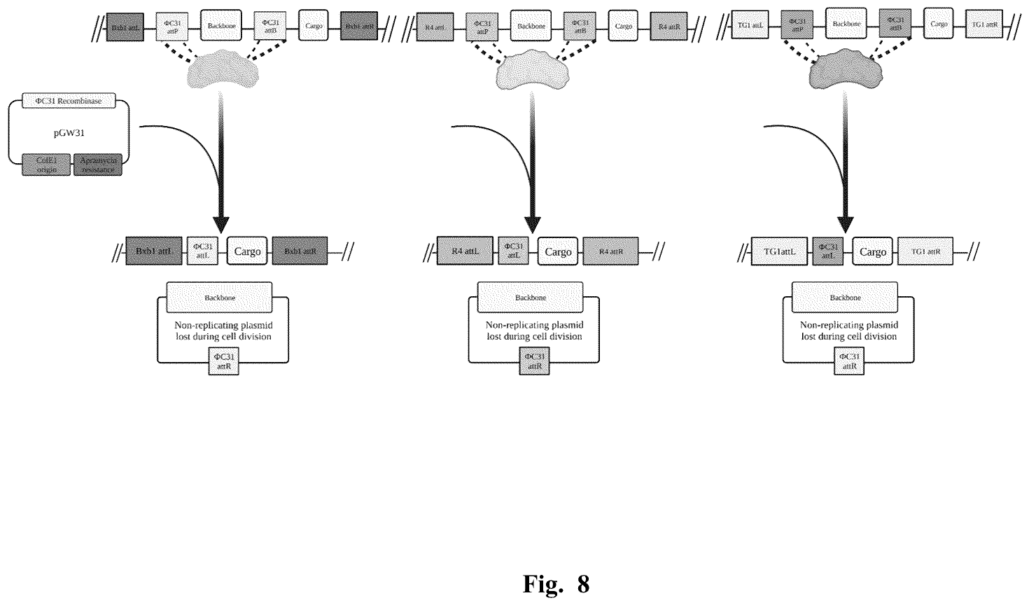

To fully realize the potential of the mSAGE cyclical process, the ability to recycle antibiotic resistance markers is critical. As described for the SAGE plasmids, all integrating plasmid backbones were flanked with a cognate pair of ϕC31 attP and attB sites which have previously been demonstrate to be orthogonal. Between the different backbones sets (based on resistance marker), orthogonal pairs that would only recombine with the att sites present on the local backbone were added. In SAGE organisms and in P. putida, single plasmid backbones could be excised with 100% efficiency. To further demonstrate the utility of mSAGE, the efficiency of the backbone removal process was examined for three plasmids which had been co-integrated (FIG. 8). A temperature sensitive $C31 recombinase expression plasmid (pGW30) was transformed into P. putida strain AG7407 where pJH204, pJH216, pJH228 had been previously integrated into the chromosome. The transformation recovery mixture was plated onto media that was selective only for pGW30 and grown at room temperature to allow plasmid replication and provide transient expression of the $C31 recombinase. Following heat curing of pGW30, twenty single isolates were grown on non-selective media and were screened via PCR to verify plasmid backbone removal from the chromosome. All 24 isolates were found to have removed the plasmid backbones at each of the three loci, demonstrating simultaneous backbone excision. This validates that the antibiotic resistance markers common between integrating plasmids can be excised rapidly and allows for subsequent rounds of DNA integration without the need for new resistance genes.

Application of mSAGE for Non-Native Catabolic Pathway Optimization

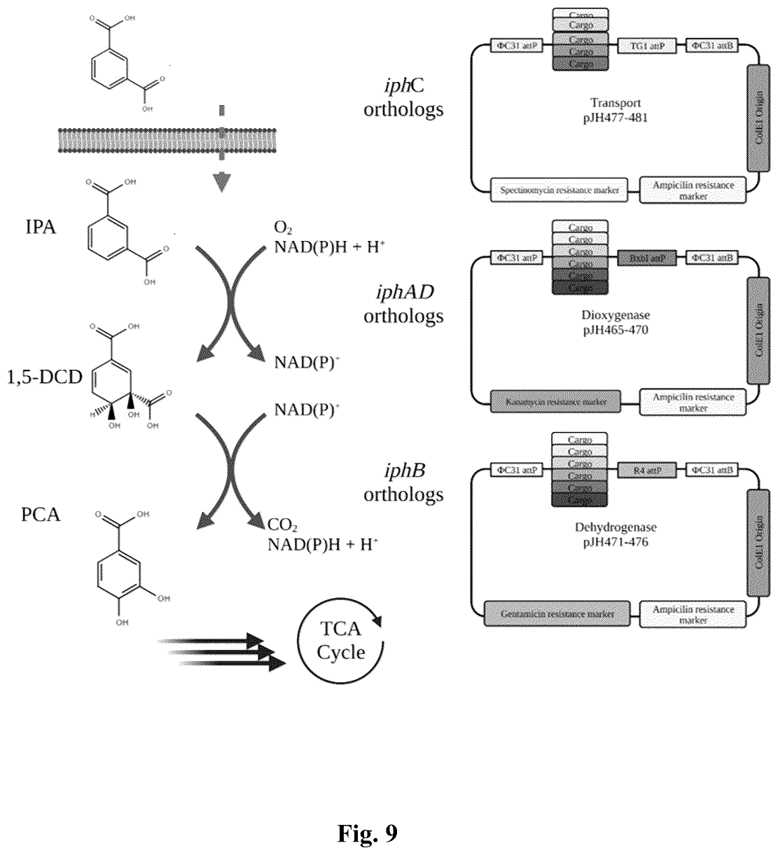

With the functionality of the mSAGE tools established, we investigated applications to demonstrate the utility of mSAGE. Isophthalic acid (IPA) is a monomer often used in the production of the common plastic polyethylene terephthalate (PET), and it can be catabolized via a three-step pathway to an aromatic intermediate protocatechuate (PCA) (FIG. 9). Aromatic catabolism is well studied in P. putida and it can utilize PCA as the sole carbon and energy source, but it cannot utilize IPA natively. Optimization of non-native catabolic pathways provides a mechanism to expand the metabolic capabilities of P. putida, and further expand its utility as a bioproduction host. Therefore, optimization of growth on IPA was selected as a demonstration target for mSAGE utility. The characterized IPA catabolic genes from Comamonas sp. Strain E6 were used as the basis for ortholog identification. Additionally, 12 bacteria that were previously isolated by the Guss lab for their ability to grow on IPA (Mand, unpublished data) were examined. Using in silico tools and mining the genomes of organisms found to consume IPA from environmental enrichments, six orthologs of iphAD (dioxygenase), iphB (dehydrogenase), four of iphC (permease) and 1 MFS transport protein were identified. Overexpression mSAGE plasmids were designed with each step in the catabolic pathway being targeting a separate landing pad with either BxbI, R4, or TG1 attP sites. The dioxygenase and dehydrogenase genes were expressed using a strong promoter and RBS (pJE111111, JER01) described in previous work. The transport genes were expressed with a medium strength promoter and RBS (pJE111411, JER01) in effort to prevent toxic effects from overproduction of membrane proteins. In total, seventeen plasmids (pJH465-481) were synthesized and sequenced.

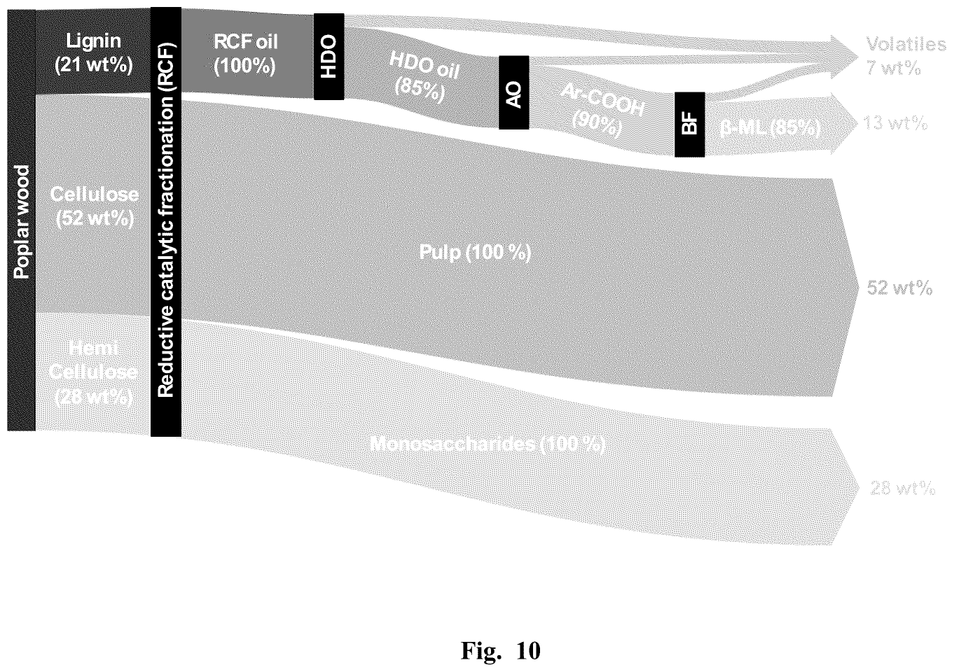

Following the generation of the ortholog integrating plasmids, growth selection was utilized to identify the best genetic combinations and create the most robust IPA catabolic pathway in P. putida. Initial attempts to pool ortholog plasmids, transform, and directly select for growth on IPA failed, likely due to the lack of sufficient protein production through the stress of recovery and adaptation to minimal media. We therefore developed a workflow to enrich and adapt the population to allow for active growth selection with IPA as the sole carbon source. FIG. 10A provides an overview of the process wherein the pooled ortholog plasmids were transformed and recovered under normal conditions (rich medium with antibiotics) and 50 μl of the recovery mixture was plated for library size quantification. Based on this plating, we estimated the total size of the transformant library to be approximately 32,000, providing more than 175× coverage of the 180 possible gene ortholog combinations. Further, twenty-four isolates were screened via PCR for the presence of all three antibiotic resistance markers. All isolates were found to have been positively integrated and possess the necessary resistance markers, indicating we had successfully integrated our library with high efficiency. The remaining recovery mixture was grown overnight in LB medium with the appropriate antibiotics to enrich the population for strains which had been successfully integrated. The entirety of the enriched population was then transferred a minimal medium with a related aromatic compound, p-coumaric acid as the sole carbon source, allowing the cells to adapt to minimal media as well as aromatic substrate catabolism. The population was then split among three shake flasks and three wells on 48 well microtiter plate. In both shake flasks and microtiter plates, growth was observed after twenty-four hours and peak density had been achieved within seventy-two hours, demonstrating the first successful engineering of P. putida to grow with IPA as the sole carbon source (FIG. 10B). The mixed population of pathway ortholog combinations was sub-cultured multiple times to allow faster-growing variants to overtake the population. We observed significant improvement in growth between the initial IPA cultures and the second sub-cultures, but observed smaller, incremental improvements over the third and fourth rounds of culturing and therefore ended the enrichment process after the fourth round of growth.

During the experimental enrichment process we collected samples from all replicates and sub-cultures to allow downstream analysis of the abundance of each IPA gene ortholog within the population. For all samples, we performed a genomic DNA extraction that could be utilized for PCR amplification and sequencing. Using a common forward primer and ortholog specific reverse primers, a pooled PCR reaction was carried out for all samples to generate amplicons of the orthologs present in the population. The amplicon libraries were barcoded and sequenced using an Illumina MiSeq instrument. Correlating the percent composition of the total reads for each sample, the dynamic change in population representation over the experimental process was examined. In FIG. 10C, a diverse population of orthologs was found in the initial minimal media outgrowth that was enriched for a select set of genes in the terminal cultures. By the third outgrowth on IPA, all cultures were found to have been enriched for only one dioxygenase gene (A. wautersii) and the MFS transporter (P. tuberum). For the dehydrogenase orthologs, two genes remained present in the population (Comamonas sp. E6 or R. bacterium). This data shows that we were able to successfully integrate a diverse population of plasmids and utilize growth selection to identify optimal IPA catabolic pathway from amongst these orthologs.

To validate this methodology, ortholog combinations mirroring those we identified through the enrichment process were re-constructed individually. Initially, two best orthologs (e.g., the A. wautersii dioxygenase and Comamonas sp. E6 dehydrogenase) were integrated into AG5577, creating all combinations of the enriched orthologs (AG9483-87). Each ortholog of the third pathway component (transporter, in this example) were then integrated to create strains harboring at least two components of the optimized pathway paired with each the individual orthologs of the third component not identified by optimization (AG9593-9620). This allowed for targeted comparison of growth characteristics for the orthologs of optimized genetic combinations against those not enriched to evaluate our selective optimization. Examination of growth profiles was carried out in the same manner as the experimental enrichment process by conducting an initial pre-culture overnight in rich medium with all antibiotics, a secondary pre-culture in minimal medium with p-coumaric acid, and the subsequent outgrowth in minimal medium with IPA as the sole carbon source. Numerous ortholog combinations could enable growth on IPA; however, none could match the observed growth of the ortholog combinations identified through the enrichment process. This further demonstrates that our selective process successfully enriched the population to the optimal ortholog combinations to achieve robust utilization of IPA as a substrate. Establishing this catabolic pathway in P. putida not only expands the biological funnel of possible substrates but also demonstrates the utility of mSAGE tools to create combinatorial libraries for metabolic engineering.

### Materials and Methods

### Plasmid Construction

All PCR reactions were carried out with Phusion® High Fidelity Polymerase (Thermo Scientific) utilizing primers synthesized by Eurofins Genomics. Synthetic DNA fragments and genes were obtained from Integrated DNA Technologies (IDT). All plasmids were constructed via T4 DNA ligation (New England Biolabs—NEB) or NEBuilder® HiFi DNA Assembly Master Mix (NEB) per the manufacturers standard protocol. All enzymes utilized for plasmid digestion were obtained from NEB. All Plasmids were transformed into NEB5-alpha F' IQ competent Escherichia coli (NEB) per the manufacturers standard protocol. Transformants were selected on the appropriate LB (Miller) plates containing either 50 mg/L Kanamycin, 15 mg/L Gentamicin, or 50 mg/L Spectinomycin and incubated at 37° C. DNA extraction was carried out using the ZymoPure Miniprep kit from Zymo Research, per the manufacturer's protocols. Plasmids were verified via Sanger sequencing performed by Eurofins Genomics. DNA synthesis services provided by Genscript. All plasmids utilized in this example are described in Table 1.

### Strain Construction

P. putida KT2440 was utilized as the wild-type parent strain for all strains constructed in this work. A base strain with three attB landing pads, AG5577 (WT KT2440 ΔPP_2876::R4_phiBT1_MR11 ΔPP_4740::BxBI_RV_phi370 ΔPP_4217/4218 intergenic::TG1_BL3_A118), was constructed via a pK18mob-sacB kanamycin resistance, sucrose resistance selection and counter-selection marker system as described previously. In summary, ˜3000 ng of plasmid DNA was electroporated into KT2440 competent cells and selected overnight on LB plates containing 50 mg/L Kanamycin at 30° C. Transformants were single colony purified onto LB plates containing 50 mg/L Kanamycin at 30° C. to ensure untransformed cells were not carried over. Counter selection was achieved by streaking colonies onto YT+25% sucrose plates (10 g/L yeast extract, 20 g/L tryptone, 250 g/L sucrose and 18 g/L agar) and incubated overnight at 30° C. The resulting colonies were screened via PCR for the excision of plasmid backbone and the insertion of desired poly-attB landing pad. PCR correct colonies were cultured overnight at 30° C., fully verified with 3 additional PCRs and mixed 1:1 with 50% glycerol for freezer storage.

To construct strains via serine recombinase integration, 100 ng of genetic cargo attP plasmids were pooled with the appropriate recombinase expression plasmid in the same quantity. Competent cells of P. putida KT2440 strains were generated from overnight LB cultures incubated at 30° C. and shaken at 225 rpm. The competent cells were then processed in similar fashion to previously described methods. Briefly, the cells are washed three times in 10% glycerol at half the original culture volume, and then resuspended in 1/50^{th} culture volume of 10% glycerol. To achieve the highest efficiencies and for repeated use, a 50 ml shake flask culture is recommended, however cultures of 5-10 ml can be utilized to generate enough cells for 1-2 transformations. The pooled plasmids were then added to 50 ul of competent cells and electroporated in the same manner previously described. Due to the high efficiency of integration, from the 1000 ul recovery mixture less than 5% was plated onto LB plates containing the appropriate combinations of 50 mg/L Kanamycin, 30 mg/L Gentamicin, 200 mg/L Spectinomycin, or 300 mg/L Streptomycin and incubated at 30° C. overnight. Single colony isolates were screened for correct integration as needed via PCR. PCR correct colonies were cultured overnight at 30° C. in LB medium containing appropriate antibiotics as concentrations previously listed. The strains were then fully verified with 3 additional PCRs and mixed 1:1 with 50% glycerol for freezer storage. All strains utilized in this example are listed in Table 1 and oligonucleotides used for strain verification are listed in Table 2.

### Culture Conditions

Generally, strain propagation of P. putida or E. coli plasmid hosts, culturing was performed in LB medium at either 30° C. or 37° C. respectively for both liquid and solid agar conditions. As required for P. putida propagation, 50 mg/L Kanamycin, 30 mg/L Gentamicin, 200 mg/L Spectinomycin and 300 mg/L Streptomycin were used individually or combined for adequate selection. For E. coli, 50 mg/L Kanamycin, 15 mg/L Gentamicin, 50 mg/L Spectinomycin, and 50 mg/L Streptomycin were used individually for adequate selection. M9 medium with 20 mM of NH₄Cl was utilized for shake flask experiments and plate reader cultures (47.8 mM Na₂HPO₄, 22 mM KH₂PO₄, 8.6 mM NaCl, 1 mM MgCl₂, 0.1 mM CaCl₂), 18 μM FeSO₄, 1×MME trace minerals, pH adjusted to 7 with KOH). In total, 1000×MME trace mineral stock solution contains per liter, 1 mL concentrated HCl, 0.5 g Na₄EDTA, 2 g FeCl₃, 0.05 g each H₃BO₃, ZnCl₂, CuCl₂·2H₂O, MnCl₂·4H₂O, (NH₄)₂MoO₄, CoCl₂·6H₂O, NiCl₂·6H₂O. The M9 medium was supplemented with 20 mM p-coumarate as a sole carbon source or 20 mM isophthalic acid as the sole carbon sources as required.

### Plasmid Backbone Excision

A recombinase expression plasmid (pGW30) hosting a temperature sensitive origin previously described to show temperature sensitivity in P. aeruginosa was constructed and shown to have similar sensitivity in P. putida KT2440. This plasmid expresses an alternate serine recombinase, phiC31, and provides resistance to 50 mg/L Apramycin at temperatures≤30° C. phiC31 was utilized due to the known recombination specificity related to two core nucleotides in the attB and attP sites, wherein if the two bases are incompatible recombination does not occur. This specificity enables the plasmid backbones to be excised without causing large scale genomic deletions as each set of backbones utilizes unique core nucleotides. To excise plasmid backbones following integration, competent cells of strains were cultured in the same manner described above. 50 ng of pGW30 is electroporated into 50 ul of competent cells and then mixed with 950 ul of SOB containing 50 mg/L Apramycin, then recovered at room temperature for 2 hours. Following recovery, 50 ul of the mixture is plated on LB media containing 50 mg/L apramycin and incubated overnight at room temperature. Single colony isolates were then cultured in 1 ml LB and incubated at 37° C. while shaking for 4 hours. 50 ul of culture is plated onto LB plates containing no antibiotic and incubated overnight at 30° C. Resulting isolates are then screened and verified for plasmid backbone removal and loss of plasmid.

### Integrase Efficiency and Accuracy Evaluation

To determine the transformation efficiency of individual recombinases, plasmids were transformed under identical conditions. Competent cells of AG5577 were prepared from a 50 ml overnight LB culture as described previously. 100 ng of the genetic cargo plasmid and the recombinase helper plasmid (e.g., pJH204 and pGW31) were combined and electroporated and recovered in the same manner described above. For plating, 1 ul and 10 ul of recovery mixture was diluted in 99 ul and 90 ul, respectively in SOB. The entire dilution was then plated on LB media containing the appropriate antibiotic concentrations and incubated overnight at 30° C. Single colony counts were performed to determine the number of transformants generated and calculate the cfu/ug DNA. For each recombinase, twenty isolates were screened via PCR to confirm accurate integration. In the same manner, co-transformation efficiencies were calculated by pooling 100 ng of three genetic cargo plasmids with 100 ng of the appropriate recombinase helper plasmids. Using the same procedure, colony counts were used to calculate the cfu/ug DNA and PCR screening was completed on twenty-four isolates to confirm accurate integration for all three plasmids.

### Isophthalic Acid Pathway Ortholog Identification and Plasmid Construction

Initial IPA degradation genes were selected from the previously described catabolic pathway in Comamonas sp. Strain E6. The gene sequences encoding iphABCD were obtained from a published genome (https://www.ncbi.nlm.nih.gov/nuccore/BBXHO1000000). The corresponding protein sequences were utilized to search and identify additional orthologs using TheSeed (https://www.theseed.org/wiki/Home_of_the_SEED). From this search, two orthologous pathways were identified in Burkholderia sp. CCGE1002 and Rhodobacterales bacterium HTCC2654. Additionally, twelve previously identified (unpublished) environmental enrichment isolates with genome sequence data were mined for orthologous IPA catabolic pathways. From theses isolates, three organisms were found to possess operons with sequence similarity and genomic context similar to Comamonas sp. E6. These isolates included another Comamonas species in C. testosteroni, along with Acidovorax wautersii and Paraburkholderia tuberum. The details of the orthologs can be found in Table 3. Using the translated protein sequences from these organisms for iphABCD orthologs, new gene sequences created to remove common restriction enzyme sites and optimize codon distributions in P. putida using Geneious software tools. Using these sequences novel plasmids were synthesized into mSAGE based backbones. Orthologs of iphAD were oriented in an operon, under the control of the pTac promoter and JER01 RBS sequence previously described and cloned into the mSAGE plasmid pJH401 digested with BamHI and XbaI. Orthologs of iphB were constructed in the same manner as iphAD plasmids excluding the alteration of the mSAGE plasmid from pJH401 to pJH413. A slightly weaker promoter variant (pJE111411) was used for the expression of the membrane associated transports gene orthologous to iphC and these genes constructed using the mSAGE plasmid pJH425. The synthesized plasmids (pJH465-481) were constructed, and sequence verified before delivery by Genscript.

### IPA Pathway Optimization

To execute the growth selection process approximately 500 ng of pJH465-481 was pooled along with 5000 ng of pGW31-38-39. From this pool, 5 ul of the mixture of plasmids was added to 50 ul of AG5577 competent cells and the electroporation was carried out as previously described. Following the normal recovery period, the entire recovery mixture was used to inoculate a 50 ml culture of LB media with 50 mg/L Kanamycin, 30 mg/L Gentamicin, 200 mg/L Spectinomycin, and 300 mg/L Streptomycin. This culture was incubated overnight at 30° C. while shaking at 225 rpm. The resulting population was then centrifuged in a 50 ml conical tube at 3000 rpm for 15 minutes to pellet the cells. The entire population was then gently resuspended in 50 ml of M9 minimal media with p-coumaric acid (ThermoFisher #A15167) as the sole carbon source. This culture was then incubated overnight at 30° C. while shaking at 225 rpm. Following this incubation, the entire population was pelleted in the same manner and the resulting cell pellet was gently resuspended in 1×M9 salts. From this suspension, 300 ul were transferred to three 50 ml M9 cultures with IPA as the sole carbon source and incubated at 30° C. while shaking at 225 rpm. Similarly, 6 ul of the suspension was transferred to a 48 well microtiter plate with 600 ul of M9 media with IPA as the sole carbon source and incubated at 30° C. in a Biotek Epoch 2 plate reader. Initially, all cultures were incubated for approximately 72 hours, then sub-cultured and incubated for 48 hours under the same conditions three more times. From all cultures, samples were collected and a whole DNA extraction was performed using a Zymo Quick-DNA Bacterial miniprep kit (Zymo, D6005). Along with this, samples were collected and mixed with 50% glycerol to be stored at −80° C.

### Ortholog Population Sequencing

To evaluate the composition of the ortholog populations over the entire experimental process, a targeted PCR amplification was performed. Using a common forward primer (oJH966) in equimolar amount to the total molar amount of 17 ortholog specific primers (oJH967-83), a pooled PCR was performed to amplify an approximately 240 bp fragment. Following amplification, the individual reactions were size selected and isolated from a 2% agarose gel using the ThermoFisher GeneJET gel extraction kit (K0691). The purified amplicons were then prepared for Illumina sequencing using the NEBNext Ultra II DNA library prep kit (NEB #E7645) per the manufacturer's instructions. Briefly, the amplicons were end repaired and ligated to NEBNext adaptors. Following ligation, the amplicons were size selected using magnetic beads. Using barcoded primers, the amplicons were again amplified with each time point and replicate using a unique barcode set. The barcoded amplicons were then sequenced using an Illumina MiSeq instrument.

TABLE 1
mSAGE Plasmids and Strain List

| Strain | Plasmid | Integrase | E. Coli | Resistance | |
|---|---|---|---|---|---|
| ID | Name | attP | Origin | Marker | Plasmid Type |
| AG4513 | pJH0204 | Bxb1 | ColE1 | Kanamycin | Target/attP Plasmid |
| AG4514 | pJH0205 | RV | ColE1 | Kanamycin | Target/attP Plasmid |
| AG4515 | pJH0206 | phi370 | ColE1 | Kanamycin | Target/attP Plasmid |
| AG4516 | pJH0207 | R4 | ColE1 | Kanamycin | Target/attP Plasmid |
| AG4517 | pJH0208 | BT | ColE1 | Kanamycin | Target/attP Plasmid |
| AG4518 | pJH0209 | MR11 | ColE1 | Kanamycin | Target/attP Plasmid |
| AG4519 | pJH0210 | TG1 | ColE1 | Kanamycin | Target/attP Plasmid |
| AG4520 | pJH0211 | BL3 | ColE1 | Kanamycin | Target/attP Plasmid |
| AG4521 | pJH0212 | A118 | ColE1 | Kanamycin | Target/attP Plasmid |
| AG4522 | pJH0213 | Bxb1 | ColA | Gentamicin | Target/attP Plasmid |
| AG4523 | pJH0214 | RV | ColA | Gentamicin | Target/attP Plasmid |
| AG4524 | pJH0215 | phi370 | ColA | Gentamicin | Target/attP Plasmid |
| AG4525 | pJH0216 | R4 | ColA | Gentamicin | Target/attP Plasmid |
| AG4526 | pJH0217 | BT | ColA | Gentamicin | Target/attP Plasmid |
| AG4527 | pJH0218 | MR11 | ColA | Gentamicin | Target/attP Plasmid |
| AG4528 | pJH0219 | TG1 | ColA | Gentamicin | Target/attP Plasmid |
| AG4529 | pJH0220 | BL3 | ColA | Gentamicin | Target/attP Plasmid |
| AG4530 | pJH0221 | A118 | ColA | Gentamicin | Target/attP Plasmid |
| AG4531 | pJH0222 | Bxb1 | CloDF13 | Spectinomycin/ | Target/attP Plasmid |
| | | | | Streptomycin | |
| AG4532 | pJH0223 | RV | CloDF13 | Spectinomycin/ | Target/attP Plasmid |
| | | | | Streptomycin | |
| AG4533 | pJH0224 | phi370 | CloDF13 | Spectinomycin/ | Target/attP Plasmid |
| | | | | Streptomycin | |
| AG4534 | pJH0225 | R4 | CloDF13 | Spectinomycin/ | Target/attP Plasmid |
| | | | | Streptomycin | |
| AG4535 | pJH0226 | BT | CloDF13 | Spectinomycin/ | Target/attP Plasmid |
| | | | | Streptomycin | |
| AG4536 | pJH0227 | MR11 | CloDF13 | Spectinomycin/ | Target/attP Plasmid |
| | | | | Streptomycin | |
| AG4537 | pJH0228 | TG1 | CloDF13 | Spectinomycin/ | Target/attP Plasmid |
| | | | | Streptomycin | |
| AG4538 | pJH0229 | BL3 | CloDF13 | Spectinomycin/ | Target/attP Plasmid |
| | | | | Streptomycin | |
| AG4539 | pJH0230 | A118 | CloDF13 | Spectinomycin/ | Target/attP Plasmid |
| | | | | Streptomycin | |
| AG3425 | pGW13 | Bxb1 | ColE1 | Apramycin | Integrase Expression |
| | | | | | TS Plasmid |
| AG3426 | pGW14 | BT | ColE1 | Apramycin | Integrase Expression |
| | | | | | TS Plasmid |
| AG3429 | pGW17 | A118 | ColE1 | Apramycin | Integrase Expression |
| | | | | | TS Plasmid |
| AG3430 | pGW18 | MR11 | ColE1 | Apramycin | Integrase Expression |
| | | | | | TS Plasmid |
| AG3432 | pGW20 | phi370 | ColE1 | Apramycin | Integrase Expression |
| | | | | | TS Plasmid |
| AG3433 | pGW21 | RV | ColE1 | Apramycin | Integrase Expression |
| | | | | | TS Plasmid |
| AG3434 | pGW22 | TG1 | ColE1 | Apramycin | Integrase Expression |
| | | | | | TS Plasmid |
| AG3435 | pGW23 | R4 | ColE1 | Apramycin | Integrase Expression |
| | | | | | TS Plasmid |
| AG3436 | pGW24 | BL3 | ColE1 | Apramycin | Integrase Expression |
| | | | | | TS Plasmid |
| AG3438 | pGW30 | phiC31 | ColE1 | Apramycin | Backbone Excision |
| | | | | | Plasmid |
| AG3439 | pGW31 | Bxb1 | ColE1 | Apramycin | Integrase Expression |
| | | | | | Suicide Vector |
| AG3440 | pGW32 | RV | ColE1 | Apramycin | Integrase Expression |
| | | | | | Suicide Vector |
| AG3441 | pGW33 | BT | ColE1 | Apramycin | Integrase Expression |
| | | | | | Suicide Vector |
| AG3443 | pGW35 | A118 | ColE1 | Apramycin | Integrase Expression |
| | | | | | Suicide Vector |
| AG3444 | pGW36 | MR11 | ColE1 | Apramycin | Integrase Expression |
| | | | | | Suicide Vector |
| AG3445 | pGW37 | phi370 | ColE1 | Apramycin | Integrase Expression |
| | | | | | Suicide Vector |
| AG3446 | pGW38 | TG1 | ColE1 | Apramycin | Integrase Expression |
| | | | | | Suicide Vector |
| AG3447 | pGW39 | R4 | ColE1 | Apramycin | Integrase Expression |
| | | | | | Suicide Vector |
| AG3448 | pGW40 | BL3 | ColE1 | Apramycin | Integrase Expression |
| | | | | | Suicide Vector |
| AG8142 | pJH401 | Bxb1 | ColE1 | Kanamycin/ | High Copy Target/ |
| | | | | Ampicillin | attP Plasmid |
| AG8143 | pJH402 | RV | ColE1 | Kanamycin/ | High Copy Target/ |
| | | | | Ampicillin | attP Plasmid |
| AG8144 | pJH403 | phi370 | ColE1 | Kanamycin/ | High Copy Target/ |
| | | | | Ampicillin | attP Plasmid |
| AG8145 | pJH404 | R4 | ColE1 | Kanamycin/ | High Copy Target/ |
| | | | | Ampicillin | attP Plasmid |
| AG8146 | pJH405 | BT | ColE1 | Kanamycin/ | High Copy Target/ |
| | | | | Ampicillin | attP Plasmid |
| AG8147 | pJH406 | MR11 | ColE1 | Kanamycin/ | High Copy Target/ |
| | | | | Ampicillin | attP Plasmid |
| AG8148 | pJH407 | TG1 | ColE1 | Kanamycin/ | High Copy Target/ |
| | | | | Ampicillin | attP Plasmid |
| AG8149 | pJH408 | BL3 | ColE1 | Kanamycin/ | High Copy Target/ |
| | | | | Ampicillin | attP Plasmid |
| AG8150 | pJH409 | A118 | ColE1 | Kanamycin/ | High Copy Target/ |
| | | | | Ampicillin | attP Plasmid |
| AG8151 | pJH410 | Bxb1 | ColE1 | Gentamicin/ | High Copy Target/ |
| | | | | Ampicillin | attP Plasmid |
| AG8152 | pJH411 | RV | ColE1 | Gentamicin/ | High Copy Target/ |
| | | | | Ampicillin | attP Plasmid |
| AG8153 | pJH412 | phi370 | ColE1 | Gentamicin/ | High Copy Target/ |
| | | | | Ampicillin | attP Plasmid |
| AG8154 | pJH413 | R4 | ColE1 | Gentamicin/ | High Copy Target/ |
| | | | | Ampicillin | attP Plasmid |
| AG8155 | pJH414 | BT | ColE1 | Gentamicin/ | High Copy Target/ |
| | | | | Ampicillin | attP Plasmid |
| AG8156 | pJH415 | MR11 | ColE1 | Gentamicin/ | High Copy Target/ |
| | | | | Ampicillin | attP Plasmid |
| AG8157 | pJH416 | TG1 | ColE1 | Gentamicin/ | High Copy Target/ |
| | | | | Ampicillin | attP Plasmid |
| AG8158 | pJH417 | BL3 | ColE1 | Gentamicin/ | High Copy Target/ |
| | | | | Ampicillin | attP Plasmid |
| AG8159 | pJH418 | A118 | ColE1 | Gentamicin/ | High Copy Target/ |
| | | | | Ampicillin | attP Plasmid |
| AG8160 | pJH419 | Bxb1 | ColE1 | Spectinomycin/ | High Copy Target/ |
| | | | | Streptomycin/ | attP Plasmid |
| | | | | Ampicillin | |
| AG8161 | pJH420 | RV | ColE1 | Spectinomycin/ | High Copy Target/ |
| | | | | Streptomycin/ | attP Plasmid |
| | | | | Ampicillin | |
| AG8162 | pJH421 | phi370 | ColE1 | Spectinomycin/ | High Copy Target/ |
| | | | | Streptomycin/ | attP Plasmid |
| | | | | Ampicillin | |
| AG8163 | pJH422 | R4 | ColE1 | Spectinomycin/ | High Copy Target/ |
| | | | | Streptomycin/ | attP Plasmid |
| | | | | Ampicillin | |
| AG3438 | pGW30 | phiC31 | ColE1 | Apramycin | Backbone Excision |
| | | | | | Plasmid |
| AG3439 | pGW31 | Bxb1 | ColE1 | Apramycin | Integrase Expression |
| | | | | | Suicide Vector |
| AG3440 | pGW32 | RV | ColE1 | Apramycin | Integrase Expression |
| | | | | | Suicide Vector |
| AG3441 | pGW33 | BT | ColE1 | Apramycin | Integrase Expression |
| | | | | | Suicide Vector |
| AG3443 | pGW35 | A118 | ColE1 | Apramycin | Integrase Expression |
| | | | | | Suicide Vector |
| AG3444 | pGW36 | MR11 | ColE1 | Apramycin | Integrase Expression |
| | | | | | Suicide Vector |
| AG3445 | pGW37 | phi370 | ColE1 | Apramycin | Integrase Expression |
| | | | | | Suicide Vector |
| AG3446 | pGW38 | TG1 | ColE1 | Apramycin | Integrase Expression |
| | | | | | Suicide Vector |
| AG3447 | pGW39 | R4 | ColE1 | Apramycin | Integrase Expression |
| | | | | | Suicide Vector |
| AG3448 | pGW40 | BL3 | ColE1 | Apramycin | Integrase Expression |
| | | | | | Suicide Vector |
| AG8142 | pJH401 | Bxb1 | ColE1 | Kanamycin/ | High Copy Target/ |
| | | | | Ampicillin | attP Plasmid |
| AG8143 | pJH402 | RV | ColE1 | Kanamycin/ | High Copy Target/ |
| | | | | Ampicillin | attP Plasmid |
| AG8144 | pJH403 | phi370 | ColE1 | Kanamycin/ | High Copy Target/ |
| | | | | Ampicillin | attP Plasmid |
| AG8145 | pJH404 | R4 | ColE1 | Kanamycin/ | High Copy Target/ |
| | | | | Ampicillin | attP Plasmid |
| AG8146 | pJH405 | BT | ColE1 | Kanamycin/ | High Copy Target/ |
| | | | | Ampicillin | attP Plasmid |
| AG8147 | pJH406 | MR11 | ColE1 | Kanamycin/ | High Copy Target/ |
| | | | | Ampicillin | attP Plasmid |
| AG8148 | pJH407 | TG1 | ColE1 | Kanamycin/ | High Copy Target/ |
| | | | | Ampicillin | attP Plasmid |
| AG8149 | pJH408 | BL3 | ColE1 | Kanamycin/ | High Copy Target/ |
| | | | | Ampicillin | attP Plasmid |
| AG8150 | pJH409 | A118 | ColE1 | Kanamycin/ | High Copy Target/ |
| | | | | Ampicillin | attP Plasmid |
| AG8151 | pJH410 | Bxb1 | ColE1 | Gentamicin/ | High Copy Target/ |
| | | | | Ampicillin | attP Plasmid |
| AG8152 | pJH411 | RV | ColE1 | Gentamicin/ | High Copy Target/ |
| | | | | Ampicillin | attP Plasmid |
| AG8153 | pJH412 | phi370 | ColE1 | Gentamicin/ | High Copy Target/ |
| | | | | Ampicillin | attP Plasmid |
| AG8154 | pJH413 | R4 | ColE1 | Gentamicin/ | High Copy Target/ |
| | | | | Ampicillin | attP Plasmid |
| AG8155 | pJH414 | BT | ColE1 | Gentamicin/ | High Copy Target/ |
| | | | | Ampicillin | attP Plasmid |
| AG8156 | pJH415 | MR11 | ColE1 | Gentamicin/ | High Copy Target/ |
| | | | | Ampicillin | attP Plasmid |
| AG8157 | pJH416 | TG1 | ColE1 | Gentamicin/ | High Copy Target/ |
| | | | | Ampicillin | attP Plasmid |
| AG8158 | pJH417 | BL3 | ColE1 | Gentamicin/ | High Copy Target/ |
| | | | | Ampicillin | attP Plasmid |
| AG8159 | pJH418 | A118 | ColE1 | Gentamicin/ | High Copy Target/ |
| | | | | Ampicillin | attP Plasmid |
| AG8160 | pJH419 | Bxb1 | ColE1 | Spectinomycin/ | High Copy Target/ |
| | | | | Streptomycin/ | attP Plasmid |
| | | | | Ampicillin | |
| AG8161 | pJH420 | RV | ColE1 | Spectinomycin/ | High Copy Target/ |
| | | | | Streptomycin/ | attP Plasmid |
| | | | | Ampicillin | |
| AG8162 | pJH421 | phi370 | ColE1 | Spectinomycin/ | High Copy Target/ |
| | | | | Streptomycin/ | attP Plasmid |
| | | | | Ampicillin | |
| AG8163 | pJH422 | R4 | ColE1 | Spectinomycin/ | High Copy Target/ |
| | | | | Streptomycin/ | attP Plasmid |
| | | | | Ampicillin | |

| Strain ID | Integrated plasmids | Description |
|---|---|---|
| AG5577 | N/A | mSAGE Base strain |
| AG7407 | pJH204-216-228 | Triple integration strain for marker excision test |
| AG9483 | AG5577 + pJH469/471 | A. wautersii iphAD, Comamonas sp. E6 iphB |
| AG9484 | AG5577 + pJH469/473 | A. wautersii iphAD, R. bacterium iphB |
| AG9485 | AG5577 + pJH471/481 | Comamonas sp. E6 iphB, P. tuberum MFS transporter |
| AG9486 | AG5577 + pJH473/481 | A. wautersii iphAD, P. tuberum MFS transporter |
| AG9487 | AG5577 + pJH469/481 | A. wautersii iphAD, P. tuberum MFS transporter |
| AG9593 | AG9483 + pJH477 | A. wautersii iphAD, Comamonas sp. E6 iphB, Comamonas |
| | | sp. E6 iphC |
| AG9594 | AG9483 + pJH478 | A. wautersii iphAD, Comamonas sp. E6 iphB, Burkholderia |
| | | sp. CCGE10002 iphC |
| AG9595 | AG9483 + pJH479 | A. wautersii iphAD, Comamonas sp. E6 iphB, A. wautersii |
| | | iphC |
| AG9596 | AG9483 + pJH480 | A. wautersii iphAD, Comamonas sp. E6 iphB, C. testosteroni |
| | | iphC |
| AG9597 | AG9483 + pJH481 | A. wautersii iphAD, Comamonas sp. E6 iphB, P. tuberum |
| | | MFS transporter |
| AG9598 | AG9484 + pJH477 | A. wautersii iphAD, R. bacterium iphB, Comamonas sp. E6 |
| | | iphC |
| AG9599 | AG9484 + pJH478 | A. wautersii iphAD, R. bacterium iphB, Burkholderia sp. |
| | | CCGE10002 iphC |
| AG9600 | AG9484 + pJH479 | A. wautersii iphAD, R. bacterium iphB, A. wautersii iphC |
| AG9601 | AG9484 + pJH480 | A. wautersii iphAD, R. bacterium iphB, C. testosteroni iphC |
| AG9602 | AG9484 + pJH481 | A. wautersii iphAD, R. bacterium iphB, P. tuberum MFS |
| | | transporter |
| AG9603 | AG9485 + pJH465 | Comamonas sp. E6 iphB, P. tuberum MFS transporter, |
| | | Comamonas sp. E6 iphAD |
| AG9604 | AG9485 + pJH466 | Comamonas sp. E6 iphB, P. tuberum MFS transporter, |
| | | Burkholderia sp. CCGE10002 iphAD |
| AG9605 | AG9485 + pJH467 | Comamonas sp. E6 iphB, P. tuberum MFS transporter, |
| | | R. bacterium iphAD |
| AG9606 | AG9485 + pJH468 | Comamonas sp. E6 iphB, P. tuberum MFS transporter, |
| | | P. tuberum iphAD |
| AG9607 | AG9485 + pJH469 | Comamonas sp. E6 iphB, P. tuberum MFS transporter, |
| | | A. wautersii iphAD |
| AG9608 | AG9485 + pJH470 | Comamonas sp. E6 iphB, P. tuberum MFS transporter, |
| | | C. testosteroni iphAD |
| AG9609 | AG9486 + pJH465 | A. wautersii iphAD, P. tuberum MFS transporter, |
| | | Comamonas sp. E6 iphAD |
| AG9610 | AG9486 + pJH466 | A. wautersii iphAD, P. tuberum MFS transporter, |
| | | Burkholderia sp. CCGE10002 iphAD |
| AG9611 | AG9486 + pJH467 | A. wautersii iphAD, P. tuberum MFS transporter, |
| | | R. bacterium iphAD |
| AG9612 | AG9486 + pJH468 | A. wautersii iphAD, P. tuberum MFS transporter, |
| | | P. tuberum iphAD |
| AG9613 | AG9486 + pJH469 | A. wautersii iphAD, P. tuberum MFS transporter, |
| | | A. wautersii iphAD |
| AG9614 | AG9486 + pJH470 | A. wautersii iphAD, P. tuberum MFS transporter, |
| | | C. testosteroni iphAD |
| AG9615 | AG9487 + pJH471 | A. wautersii iphAD, P. tuberum MFS transporter, |
| | | Comamonas sp. E6 iphB |
| AG9616 | AG9487 + pJH472 | A. wautersii iphAD, P. tuberum MFS transporter, |
| | | Burkholderia sp. CCGE10002 iphB |
| AG9617 | AG9487 + pJH473 | A. wautersii iphAD, P. tuberum MFS transporter, |
| | | R. bacterium iphB |
| AG9618 | AG9487 + pJH474 | A. wautersii iphAD, P. tuberum MFS transporter, |
| | | P. tuberum iphB |
| AG9619 | AG9487 + pJH475 | A. wautersii iphAD, P. tuberum MFS transporter, |
| | | A. wautersii iphB |
| AG9620 | AG9487 + pJH476 | A. wautersii iphAD, P. tuberum MFS transporter, |
| | | C. testosteroni iphB |

TABLE 2
mSAGE Oligonucleotides

| Primer | | | |
|---|---|---|---|
| Name | 5′-3′ Sequence | Description | Purpose |
| oJH0716 | TCCCTTGTCCAG | Forward primer targeting pJH204-212 | Plasmid |
| | ATAGCCCA | backbones | integration |
| | | | Screen PCR |
| oJH0717 | TGACGCTCAGTG | Reverse primer targeting pJH204-212 | Plasmid |
| | GAACGAAA | backbones | integration |
| | | | Screen PCR |
| oJH0718 | CTGTGTGAGCTC | Forward primer targeting pJH213-221 | Plasmid |
| | ACAATTCC | backbones | integration |
| | | | Screen PCR |
| oJH0719 | TTCTAGGACGTT | Reverse primer targeting pJH213-221 | Plasmid |
| | TCTGCGCA | backbones | integration |
| | | | Screen PCR |
| oJH0720 | ACTGGGTTCGTG | Forward primer targeting pJH222-230 | Plasmid |
| | CCTTCATC | backbones | integration |
| | | | Screen PCR |
| oJH0721 | TTTCTACTGAAC | Reverse primer targeting pJH222-230 | Plasmid |
| | CGCGCATG | backbones | integration |
| | | | Screen PCR |
| oJH0754 | CGACGATAGTGG | Upstream Flank targeting KT2440 | Plasmid |
| | CAGCATG | ΔPP_4740 poly attB landing pad region | integration |
| | | | Screen PCR |
| oJH0757 | AACGGCGTCAAC | Upstream Flank targeting ΔPP_2876 | Plasmid |
| | CATTTTGT | poly attB landing pad region | integration |
| | | | Screen PCR |
| oJH0758 | GCAAGGCCAGG | Downstream Flank targeting ΔPP_2876 | Plasmid |
| | GTGAAATTG | poly attB landing pad region | integration |
| | | | Screen PCR |
| oJH0759 | CCGTACGACGTG | Upstream Flank targeting APP_4217/18 | Plasmid |
| | TATGGTGA | intergenic poly attB landing pad | integration |
| | | region | Screen PCR |
| oJH0760 | CATGACCAGGGT | Downstream Flank targeting | Plasmid |
| | GTCGCTTA | ΔPP_4217/18 intergenic poly attB | integration |
| | | landing pad region | Screen PCR |
| oJH0761 | GCCACTCCCAAA | Forward primer targeting BxbI and RV1 | Plasmid |
| | ATTCTGCC | poly attB 1 region | integration |
| | | | Screen PCR |
| oJH0762 | GGCAGAATTTTG | Reverse primer targeting BxBI and RV1 | Plasmid |
| | GGAGTGGC | poly attB 1 region | integration |
| | | | Screen PCR |
| oJH0763 | GTTCGCTCCACT | Forward primer targeting RV1 and | Plasmid |
| | AAAGTTGT | phi370 poly attB 1 region | integration |
| | | | Screen PCR |
| oJH0764 | ACAACTTTAGTG | Reverse primer targeting RV1 and | Plasmid |
| | GAGCGAAC | phi370 poly attB 1 region | integration |
| | | | Screen PCR |
| oJH0765 | ACTCCCCAATAT | Forward primer targeting R4 and | Plasmid |
| | GTGAGCA | phiBT1 poly attB 2 region | integration |
| | | | Screen PCR |
| oJH0766 | CCCGTGCAACAT | Reverse primer targeting R4 and | Plasmid |
| | CAGATGC | phiBT1 poly attB 2 region | integration |
| | | | Screen PCR |
| oJH0767 | AGAGTTGTCAGT | Forward primer targeting phiBT1 and | Plasmid |
| | TAGCTCGT | MR11 poly attB 2 region | integration |
| | | | Screen PCR |
| oJH0768 | ACCTGAACGAGC | Reverse primer targeting phiBT1 and | Plasmid |
| | TAACTGAC | MR11 poly attB 2 region | integration |
| | | | Screen PCR |
| oJH0769 | AACATGACTGCT | Forward primer targeting TG1 and BL3 | Plasmid |
| | GTCGGC | poly attB 3 region | integration |
| | | | Screen PCR |
| oJH0770 | GGCCCTTGTGCC | Reverse primer targeting TG1 and BL3 | Plasmid |
| | GACAG | poly attB 3 region | integration |
| | | | Screen PCR |
| oJH0771 | CGTCCAAGCGGA | Forward primer targeting BL3 and | Plasmid |
| | TGCAATG | A118 poly attB 3 region | integration |
| | | | Screen PCR |
| oJH0772 | GGATCGCATTGC | Reverse primer targeting BL3 and A118 | Plasmid |
| | ATCCGC | poly attB 3 region | integration |
| | | | Screen PCR |
| oJH0895 | CTGGAGTGGTGC | Gene Target Screen forward primer- | Plasmid |
| | ACCCTG | pJH465 | integration |
| | | | Screen PCR |
| oJH0896 | GGCTGCCACTTG | Gene Target Screen reverse primer- | Plasmid |
| | TACGGT | pJH465 | integration |
| | | | Screen PCR |
| oJH0897 | ACCACGGCCTCA | Gene Target Screen forward primer- | Plasmid |
| | AGTTCG | pJH466 | integration |
| | | | Screen PCR |
| oJH0898 | CGGTATCGAGCA | Gene Target Screen reverse primer- | Plasmid |
| | CGGCAT | pJH466 | integration |
| | | | Screen PCR |
| oJH0899 | GACCACATCTGC | Gene Target Screen forward primer- | Plasmid |
| | CCCCAC | pJH467 | integration |
| | | | Screen PCR |
| oJH0900 | GTAACGCCGATA | Gene Target Screen reverse primer- | Plasmid |
| | CCCCCG | pJH467 | integration |
| | | | Screen PCR |
| oJH0901 | ACCACGGCCTGA | Gene Target Screen forward primer- | Plasmid |
| | AGTTCG | pJH468 | integration |
| | | | Screen PCR |
| oJH0904 | CCCAGCGCAGTA | Gene Target Screen reverse primer- | Plasmid |
| | CACCAA | pJH469 | integration |
| | | | Screen PCR |
| oJH0905 | CGTCCTCGCCCC | Gene Target Screen forward primer- | Plasmid |
| | AAAGTT | pJH470 | integration |
| | | | Screen PCR |
| oJH0906 | ATGGCCCTGCAG | Gene Target Screen reverse primer- | Plasmid |
| | CAAGTT | pJH470 | integration |
| | | | Screen PCR |
| oJH0907 | GCAGGGATCGGG | Gene Target Screen forward primer- | Plasmid |
| | GCAG | pJH471 | integration |
| | | | Screen PCR |
| oJH0908 | TGAGGGTCGTAC | Gene Target Screen reverse primer- | Plasmid |
| | CGGTGA | pJH471 | integration |
| | | | Screen PCR |
| oJH0909 | CAGGTGCTTCGA | Gene Target Screen forward primer- | Plasmid |
| | AGGGCA | pJH472 | integration |
| | | | Screen PCR |
| oJH0910 | AGAACACCTGCA | Gene Target Screen reverse primer- | Plasmid |
| | TGCGCT | pJH472 | integration |
| | | | Screen PCR |
| oJH0911 | ACGAACTCAAGA | Gene Target Screen forward primer- | Plasmid |
| | CCGCCG | pJH473 | integration |
| | | | Screen PCR |
| oJH0912 | CGTGTCTTGACG | Gene Target Screen reverse primer- | Plasmid |
| | CTGGGT | pJH473 | integration |
| | | | Screen PCR |
| oJH0913 | CCGTACTGCTCG | Gene Target Screen forward primer- | Plasmid |
| | TGGACG | pJH474 | integration |
| | | | Screen PCR |
| oJH0914 | CTCGAGGCCAGC | Gene Target Screen reverse primer- | Plasmid |
| | CATACG | pJH474 | integration |
| | | | Screen PCR |
| oJH0915 | GAGGCTATCCTG | Gene Target Screen forward primer- | Plasmid |
| | CAGGCG | pJH475 | integration |
| | | | Screen PCR |
| oJH0916 | ATTTCCTCCGGG | Gene Target Screen reverse primer- | Plasmid |
| | CTTGCC | pJH475 | integration |
| | | | Screen PCR |
| oJH0917 | TGTGTTCTGCGC | Gene Target Screen forward primer- | Plasmid |
| | TGAGGG | pJH476 | integration |
| | | | Screen PCR |
| oJH0918 | TCTGTCAGTGTG | Gene Target Screen reverse primer- | Plasmid |
| | CTGCCG | pJH476 | integration |
| | | | Screen PCR |
| oJH0919 | CCTCGGCCAACC | Gene Target Screen forward primer- | Plasmid |
| | ATTCGT | pJH477 | integration |
| | | | Screen PCR |
| oJH0920 | GGTCGGGATGGT | Gene Target Screen reverse primer- | Plasmid |
| | AGGGGT | pJH477 | integration |
| | | | Screen PCR |
| oJH0921 | CGTAATCCAGGC | Gene Target Screen forward primer- | Plasmid |
| | CGCGAT | pJH478 | integration |
| | | | Screen PCR |
| oJH0922 | AGCGAACCCACC | Gene Target Screen reverse primer- | Plasmid |
| | AGCATC | pJH478 | integration |
| | | | Screen PCR |
| oJH0923 | TCGCACGTATGA | Gene Target Screen forward primer- | Plasmid |
| | TCGCCC | pJH479 | integration |
| | | | Screen PCR |
| oJH0924 | AACTGCTTGGGG | Gene Target Screen reverse primer- | Plasmid |
| | CTCGAC | pJH479 | integration |
| | | | Screen PCR |
| oJH0925 | CGCATCGTGGTC | Gene Target Screen forward primer- | Plasmid |
| | CCCATT | pJH480 | integration |
| | | | Screen PCR |
| oJH0926 | GGGTGCTGGCAA | Gene Target Screen reverse primer- | Plasmid |
| | TCGTCT | pJH480 | integration |
| | | | Screen PCR |
| oJH0927 | TGGGCATCTCCG | Gene Target Screen forward primer- | Plasmid |
| | TCTCGA | pJH481 | integration |
| | | | Screen PCR |
| oJH0928 | GATCGGCGCGAC | Gene Target Screen reverse primer- | Plasmid |
| | CAAGTA | pJH481 | integration |
| | | | Screen PCR |
| oJH0966 | GTCCGGATCCCT | Froward primer for IPA strain sequence | Pooled PCR |
| | ATGGAGGTCAGG | screen | for Population |
| | | | Tracking |
| oJH0967 | GTCGGCCTCAGC | Reverse primer for IPA strain seq | Pooled PCR |
| | GATCTCGC | screen-pJH465 | for Population |
| | | | Tracking |
| oJH0968 | GGCTCGGCGATT | Reverse primer for IPA strain seq | Pooled PCR |
| | TCGCTCGC | screen-pJH466 | for Population |
| | | | Tracking |
| oJH0969 | CCTTCTCCAGGT | Reverse primer for IPA strain seq | Pooled PCR |
| | CGCTCGACG | screen-pJH467 | for Population |
| | | | Tracking |
| oJH0970 | CTGGCTCCTCAA | Reverse primer for IPA strain seq | Pooled PCR |
| | TTTCGGACG | screen-pJH468 | for Population |
| | | | Tracking |
| oJH0971 | CGCATCCGGGAT | Reverse primer for IPA strain seq | Pooled PCR |
| | TTCGCTGC | screen-pJH469 | for Population |
| | | | Tracking |
| oJH0972 | GGCCTCCGCGAT | Reverse primer for IPA strain seq | Pooled PCR |
| | TTCGGACG | screen-pJH470 | for Population |
| | | | Tracking |
| oJH0973 | GTCGACCATCAG | Reverse primer for IPA strain seq | Pooled PCR |
| | CACGGCCG | screen-pJH471 | for Population |
| | | | Tracking |
| oJH0974 | GCGCTACCAGGG | Reverse primer for IPA strain seq | Pooled PCR |
| | TGACCTGC | screen-pJH472 | for Population |
| | | | Tracking |
| oJH0975 | ACCAACACGACA | Reverse primer for IPA strain seq | Pooled PCR |
| | GCGGCCCC | screen-pJH473 | for Population |
| | | | Tracking |
| oJH0976 | ACGAGCAGTACG | Reverse primer for IPA strain seq | Pooled PCR |
| | GCAGCGCC | screen-pJH474 | for Population |
| | | | Tracking |
| oJH0977 | GACCATCAGCAC | Reverse primer for IPA strain seq | Pooled PCR |
| | TGCCGCGC | screen-pJH475 | for Population |
| | | | Tracking |
| oJH0978 | CCAGCACGACAG | Reverse primer for IPA strain seq | Pooled PCR |
| | CCGCGCC | screen-pJH476 | for Population |
| | | | Tracking |
| oJH0979 | CGGACCGGTTTG | Reverse primer for IPA strain seq | Pooled PCR |
| | GTCGGGCG | screen-pJH477 | for Population |
| | | | Tracking |
| oJH0980 | GAAGAGGGCAA | Reverse primer for IPA strain seq | Pooled PCR |
| | CCCACCGG | screen-pJH478 | for Population |
| | | | Tracking |
| oJH0981 | TACGGACCGGCT | Reverse primer for IPA strain seq | Pooled PCR |
| | TCGTCGGC | screen-pJH479 | for Population |
| | | | Tracking |
| oJH0982 | CGTACCGGTTTT | Reverse primer for IPA strain seq | Pooled PCR |
| | GTTGGGCGCC | screen-pJH480 | for Population |
| | | | Tracking |
| oJH0983 | CAGGGCAACCCA | Reverse primer for IPA strain seq | Pooled PCR |
| | GCGGTACG | screen-pJH481 | for Population |
| | | | Tracking |

TABLE 3
IPA pathway ortholog sequences

| | Source | |
|---|---|---|
| Name | Organism | Sequence |
| iphA | Comamonas | MNKEMSETLTRVGPNTRMGNLLRRYWVPALMSSEIAEADG |
| | sp. E6 | PQVRVQLLGEKLLAFRNTDGKACLISEFCSHRGVSLYFGRNE |
| | | ENGIRCAYHGVKFDGDGQCVDVPSSPQSCARMHIKGYPCVE |
| | | RGGIVWTYMGPEEHKPSPPELEWCTLPPEHVFVSKRLQYSN |
| | | WLQAMEGGIDTAHVSYVHRFEVDTDPMHQGVKALDYIKAD |
| | | GNVKFEIEQTPFGLSLFGRRNGEPDSYYWRITQWLFPWFTLI |
| | | APFGQHALGGHVWVPIDDHNCWAWSINWQPDQPLTEEERT |
| | | SMEEGKGIHVEYEAPGSFIPKANRNNDYGMDRVAQREERSY |
| | | SGIFGFSAQDYSLQESMGPIQDHAAERLLPTDKAIVMARRML |
| | | NEAALGLEQGETPPALDASEQHVRPAGVLLPRDQDPVAWAR |
| | | EELADATKKPVFSL* |
| iphA | Burkholderia | MDKNMSETLVRTGPGTAMGNLMRRYWVPVLLASEIAEPDC |
| | sp. | PPVRVQILGEKLLAFRDSEGQPALIDEFCSHRGVSLYFGRNEE |
| | CCGE10002 | NGIRCSYHGLKFDRNGNCVEVPSAPQACKHMGITAYPCIERA |
| | | GIVWAYMGPKDRQPAPPDLEWCNLPDSHVFVSKRLQESNYL |
| | | QAMEGGIDTSHVSYVHRYEVDDDPMHQGTKALDYIKADGN |
| | | VIFEIEKQPFGLTLFGRRNGEPDSYYWRVTQWLFPWYTLIPPF |
| | | GDHSLAGHVWVPIDDHSCWAWSINFRPDRPLDEQELADLNA |
| | | GKGIHCEYEEGGSFRPKANKDNDYLIDRKAQKEKRAYSGVF |
| | | GFAMQDASLQESMGPIQDHSKEKLLPTDRAIVMARRMMYE |
| | | AATALVPDTDPPAIDADQQRVRAAGVLLPRDQKPQEWAVIH |
| | | LHDGKDQPIYTI |
| iphA | Rhodobacterales | MLSHEDNETLVRVGPGTTMGDMMRLYWLPFMASSDLEKD |
| | bacterium | GQPQTVKLLDETLIVFRDSEGRVGLVDHICPHRGAPLVFGRN |
| | HTCC2654 | EDCGLRCVYHGWKFDVDGNVADMPAEPPRSRLKDRVKIKS |
| | | YPCVERGGVVWTYMGDEPDESRPALPSFEWNMVPEENVVV |
| | | TFRVQECNWLQALEGEIDSAHAPILHGRIDDGGSINQWVAKR |
| | | DLRPTFECMRQDFGMSIASRRVLDDDTLYWRVNQFVMPFFS |
| | | LVPPQSNEFYELSGHAWVPIDDENTLCIMFSYRPDEPLHPKSR |
| | | KVFLDGHNGRETGHPSREGFDDQGAQVPFGRYVSKYRRETG |
| | | WLFDNEAQKTTWFSGLPGLWVQDAACQSGVLRVYDRTREH |
| | | LCTSDTGIAMTRRMLLETAHAFRDSGKKPDRFDDPDLYLVR |
| | | AVSLKLPKDLPWAEAGKAPMTAKVGEGLGYEL |
| iphA | Paraburkholderia | MDKNMSETLVRTGPGTAMGNLMRRYWVPVLLSSEIEEPDCP |
| | tuberum | PVRVQILGEKLLAFRDSEGQPALIDEFCSHRGVSLFFGRNEEN |
| | | GIRCSYHGLKFDRNGNCVEVPSAPQACKHMGITAYPCIERAG |
| | | IVWTYMGPKDRQPAPPDLEWVSLPASHVFVSKRLQESNYLQ |
| | | AMEGGIDTSHVSYVHRYEVDDDPMHQGTKALDYIKADGNV |
| | | IFEIEKQDFGLTLFGRRNGEPDSYYWRVTQWLFPWYTLIPPF |
| | | GDHSLAGHVWVPIDDHSCWAWSVNFRPDRPLDEQELADLN |
| | | AGKGIHCEYEEGGKFRPKANKDNDYLIDRQAQKDKRAYSG |
| | | VFGFAMQDASLQESMGPIQDHSKEKLLPTDRAIVMARRMLY |
| | | EAATALVPDTDPPAIDANQQRVRAAGVLLPRDQKPQEWAVI |
| | | HLHDGKDQPIYSI* |
| iphA | Acidovorax | MNKEMSETLTRVGPGTRMGNLMRRYWVPALACSEIPDADG |
| | wautersii | PQVRVQLLGEKLLAFRNSDGQACLIGEFCSHRGVSLYFGRNE |
| | | QNGIRCAYHGVKFDGMGQCVDVPSSPQACSRMHIKGYPCVE |
| | | RGGIVWAYMGPADQQPAPPELEWCTLPPEHVFVSKRLQYSN |
| | | WLQAMEGGIDTAHVSYVHRFEVDTDPMHQGVKALDYIKAD |
| | | GNVKFEIEQTPFGLSLFGRRNGEEDSYYWRITQYLFPWFTLIA |
| | | PFGEHALGGHVWVPIDDHHCWAWSINWQPGQPLTTEERTA |
| | | MEEGKGIHVEYEAPGSFIPKANRDNDYGMDRVAQKEERSYS |
| | | GIFGFSAQDYSLQESMGSIQDHEAEKLLPTDKAIVMARRMLH |
| | | EAALGLEQGQTPPALDAREQHVRPAGVLLPRDQDPVAWAR |
| | | EELADATKKPVFSL* |
| iphA | Comamonas | MNKEMSETLTRVGPNTRMGNLLRRYWVPALMSSEIAEADG |
| | testosteroni | PQVRVQLLGEKLLAFRNTDGKACLISEFCSHRGVSLYFGRNE |
| | | ENGIRCAYHGVKFDGDGQCVDVPSSPQSCARMHIKGYPCVE |
| | | RGGIVWTYMGPEEHKPSPPELEWCTLPPEHVFVSKRLQYSN |
| | | WLQAMEGGIDTAHVSYVHRFEVDTDPMHQGVKALDYIKAD |
| | | GNVKFEIEQTPFGLSLFGRRNGEPDSYYWRITQWLFPWFTLI |
| | | APFGNHALGGHVWVPIDDHNCWAWSINWQPDQPLTKEERQ |
| | | SMEEGKGIHVEYEEPGSFIPKANRNNDYGMDRVAQREERSY |
| | | SGIFGFSAQDYSLQESMGPIQDHAAERLLPTDKAIVMARRML |
| | | NEAALGLEQGETPPALDASEQHVRPAGVLLPRDQDPVAWAR |
| | | EELADATKKPVFSL* |
| iphB | Comamonas | MSESRMAGRTALITGAGAGIGAAASHLFCQEGAAVLMVDA |
| | sp. E6 | NAEALERTREAILQAVPGARLACATADVSDEAAAAAAVGQ |
| | | CVQQWGGLDTLVNNAAMRNYSAAADATAAEWQAMVGVN |
| | | LVGMSNYCRAALPALRQSGTGSIVNVSSCYAVTGRKGMAL |
| | | YDATKAAQLAYTRSLAFEEAAHGVRANAVCPGSTLTDFHV |
| | | GRARNAGKSVEQLRTERKDTSLIGRWASPEEIAWPILWLASS |
| | | EASFITGTTLMVDGGLHIM* |
| iphB | Burkholderia | MKQRKRLEDKVALITGGGGGIGAATARVFCAEGAAVVLVD |
| | sp. | ANQEALARVADELKTADPSARVETFAADVSNDADAMRAVQ |
| | CCGE10002 | LAADSFGRLDVLVNNAAMRNYSALADATPAEWQAMVSVN |
| | | LVGTSNYCSAALPFLRRAGRASIVNVSSCYAVTGRKGMGLY |
| | | DATKAGMLAMTRTLAFEETANGVRVNAVCPGSTLTEFHVN |
| | | RAIASGKSVEVLKTQRQDTSLIGRWASPEEIAWPILWFASDE |
| | | ASYITGTTLMVDGGLSAM |
| iphB | Rhodobacterales | MDLNLSGLNALVTGASKGIGLATARTLAQEGVQVTLVARDC |
| | bacterium | DRLADAAAQIEGDTGLRPDVVAADLATREGVERVAARETPV |
| | HTCC2654 | DILVNNAGAIPPGDLASIDEDRWRAAWDLKVFGYINMTRAL |
| | | APRIAERGGVIVNVIGAGGEALSADYICGAVGNAALMAFTR |
| | | AYAKQFNSMGGRIVGLNPGLVATERMQVFLKSRAAAELGD |
| | | EGRADELTRGLPYGRAADPQEVADAVAFLASPRSGYTNGTI |
| | | LSLHGGG |
| iphB | Paraburkholderia | MQQRKRLEDKVALITGGGGGIGAATARVFCAQGAAVVLVD |
| | tuberum | ANPEALARVADELKTADPSARVETFAADVSHEGDAARAIQL |
| | | AADSFGKLDVLVNNAAMRNYSALADATPAEWQAMVSVNLI |
| | | GTSNYCRAALPFLRRAGRASIVNVSSCYAVTGRKGMGLYDT |
| | | TKAGMLAMTRTLAFEEAAHGVRANAVCPGSTLTDFHVNRA |
| | | VAAGRSVEVLKTQRQDTSLIGRWASPEEIAWPILWFASDEAS |
| | | YITGTTLMVDGGLSAM* |
| iphB | Acidovorax | MGEQRLAGRTALITGAGAGIGAAAAHLFCREGAAVLLVDA |
| | wautersii | NGDALERTRQAIADAVPGARLACATADVSDEAAALAAVQQ |
| | | CTGAWGGLDILVNNAAMRNYSAAADATAAEWQAMVGVN |
| | | LVGMSHYCRAALPALRQSGAGSIVNVSSCYAVTGRKGMAL |
| | | YDATKAAQLAYTRTLAFEEAPHGVRANAVCPGSTLTDFHVG |
| | | RAQTAGKSVDQLRTERKDSSLIGRWASPEEIAWPIVWLASSE |
| | | ASFITGTTLMVDGGLHIM* |
| iphB | Comamonas | MSESRMAGRTALITGAGAGIGAAASHLFCKEGAAVLMVDA |
| | testosteroni | NAEALERTREAILQAVPGARLACVMADVSDESAAAAAVGQ |
| | | CVQQWGGLDTLVNNAAMRNYSAAADATAAEWQAMVGVN |
| | | LVGMSNYCRAALPALRQSGTGSIVNVSSCYAVTGRKGMAL |
| | | YDATKAAQLAYTRSLAFEEAAHGVRANAVCPGSTLTDFHV |
| | | GRARNAGKSVEQLRTERKDTSLIGRWASPEEIAWPIFWLASR |
| | | EASFITGTTLMVDGGLHIM* |
| iphC | Comamonas | MKFPMTLKAALVAGACAATAAWGQAAAAPEWRPTKPVRI |
| | sp. E6 | VVPITGSTNDVLARLIAPKLQEALGQPFVVENKPGAGGNIGA |
| | | YEVSRSVPDGHTLLIGYNGPLAINVTLFDKMPYDPLKDLAPIT |
| | | LAVKSPQYLVVNPKAEFKDVKDFIAKAKANPSKYSYGSVAM |
| | | GSASHLTMEMMKSAAGFQMTHVPYKGAGPAVTDLIAGNVQ |
| | | SGFFVPGNVQGFVKEGRLKLLASTGAKRFPSTPTIPTLAESGL |
| | | KDFEATSWIGLLAPAGTPPAVINTYHQAMVRILNSPDVRKHL |
| | | DEMEFETIASTPKQFSDWIATEIGRWGKVIKATNAKAD* |
| iphC | Burkholderia | MEKNLASSQGMSASVWSSNGAREASSADTNPYRWVALFIV |
| | sp. | WAAFLLSYVVRVAWSTVAAPVGASLGISVSMLGAFVTAFY |
| | CCGE10002 | AGYVLANVVGGMLTDLLGGRAMLTLALLPLGVLTFTFGYT |
| | | HSLAFGIVIQAAMGFAAGADYSAGMKIIPAWFTRDRGRAMG |
| | | LYTTATSLAVVIANAVVPSFSARHGWSNAFHMLGIVTFAWG |
| | | IVTLLLLKNRPSNEAKPARNSLQDMLGLLRNRNLIALSIAGC |
| | | GGLWATVGFAAWGNALMTRQYGIAPIVAGSIVASFGIGAVI |
| | | AKPTLGWISDLPGVSRRMMSIGCLIAFTIALLVFGYCSTAHQF |
| | | YLVAPILGAFSFGYLPVLMAQVSDASGKRLAGASAGWTNAI |
| | | WQSGSAVSPMLVGSLYGASHSFLLALITLAIGPAIAVVAMFF |
| | | VNPQIARE |
| iphC | Acidovorax | MKFPMKLAAGLALLACASSTAWAQAAASDWRPTKPVRIVV |
| | wautersii | PITGSTNDVLARMIAPKLQEALGQPFFVDNKPGAGGNIGAAE |
| | | VARSAPDGHTLLIGYNGPLAINVTLFDKMPYDPQKDLTPITL |
| | | AVKSPQYLVVNPDTGVTDVKDFIAKAKADPKKFAYGSVAM |
| | | GSASHLTMEMMKSAAGFQATHVPYRGAGPAVTDLIAGNIQ |
| | | AGFFVPGNVQGFVKEGRLKLLASTGPKRFPSTPDIPTLAESGL |
| | | KDFEATSWIGLLAPANTPPAIVSSYHQAMVRILNSPDITKRLQ |
| | | EMEFEVVASSPKQFSDWIGTEITRWGKVIKATGAKAE* |
| iphC | Comamonas | MKFPMTLKAALVAGACAATAAWGQAAAAPEWRPTKPVRI |
| | testosteroni | VVPITGSTNDVLARLIAPKLQEALGQPFVVENKPGAGGNIGA |
| | | YEVSRSVPDGHTLLIGYNGPLAINVTLFDKMPYDPLKDLAPIT |
| | | LAVKSPQYLVVNPKAGFKDVKDFIAKAKANPSKYSYGSVA |
| | | MGSASHLTMEMMKSAAGFQMTHVPYKGAGPAVTDLIAGN |
| | | VQSGFFVPGNVQGFVKEGRLKLLASTGAKRFPSTPTIPTLAES |
| | | GLKDFEATSWIGLLAPAGTPPAVINTYHQAMVRILNSPDVRK |
| | | HLDEMEFETIASTPKQFNDWIATEIGRWGKVIKATNAKAD* |
| iphD | Comamonas | MASTYLQARVHQMRYEAAGTLSVELRPLVVAEEFAQPVQA |
| | sp. E6 | GAHIDLHLADGLIRSYSLINPGERHRYVVAVSLDPASRGGSR |
| | | FVHEKLRVGQAIQIGGPRNHFPLDETASHSVLVAGGIGITPVL |
| | | SMLRKLHALGRTAHLIYCASSRENAAFVPEIEAIAAQAGGRV |
| | | TVDWHFKDEKGVRADLYSLLQGHAEGAHFYACGPLVFLAS |
| | | YEDSCQKLGLAHVHLERFAAAPLAAPQTPEVGYAVELRRTG |
| | | KTVQVAAGTSLLDTLINAGMNPEYSCREGVCGACEVRVISG |
| | | DVDHRDQILSEQERAANKSMMICVSGCRSGNLVLDC* |
| iphD | Burkholderia | MNMTDSTLMMRVAARRDEADGIAGFEFVDADGRELPPFEA |
| | sp. | GAHIDVYVPGGPVRQYSLCNAPHERHRYQIAVLRDANSRGG |
| | CCGE10002 | SQRMHDAVNEGDAIHIGVPRNHFPLARHDAKPLLLAGGIGV |
| | | TPILCMAEQLAAKGAAFDMHYCARSKSRAAFVERIAASSWA |
| | | DNVQYHFDDEHGMLDLNALLTGGADRHLYVCGPQGFMNA |
| | | VLDTARSLGWSDDRLHYEYFAAAQPSGDGESFDVRLARSGR |
| | | VVSIAADCTVTQALAAAGVDVPVSCEQGICGTCITRVLDGEP |
| | | DHRDLYLSPEEQARNDQFLPCCSRAKSRVLVLDL |
| iphD | Rhodobacterales | MSKGADIPVRVRQMRYEADTILSVEFETLDGRDLPVAAPGS |
| | bacterium | HIDVALGKDFRRSYSLTREITGGPSCTVAIHRDPKSKGGSAY |
| | HTCC2654 | VHETLRVGDKTVISRPKNNFPLDENADLSVLIAGGIGVTPVL |
| | | CMIRRLVATGKAWKLHYAARSRSAAAFRDELDALEQASDG |
| | | KGKLHYHLDDEQNGALVDIGGILTANPTAHFYCCGPEAMLA |
| | | AYERAARGVPRDQVHVEYFSSSEEVARDGGFEVVLDRSGKT |
| | | IVVEPGQTILDALIANGVHVPFSCAEGTCGTCETDVIEGRPDH |
| | | RDIILTDEERAESKTMMICCSGSKSARLVLDI |
| iphD | Paraburkholderia | MNMTDSTLMVRVAARRDEADDIAGFEFVDVDGRELPPFEA |
| | tuberum | GAHIDVYVPGGPVRQYSLCNAPGERHRYQIAVLRDAGSRGG |
| | | SQRMHDAVNPGDAIRIGVPRNHFPLAPHDAQPLLLAGGIGVT |
| | | PILCMAEQLAATGAAFEMHYCARSKSRAAFVERIAASPWAA |
| | | RVQYHFDDEHGVLDLRALLAGVSAGRHLYVCGPQGFMNAV |
| | | LDTARSLGWSDDRLHYEYFAAAQPAGDGGSFDVRLARSGR |
| | | VVPVAADCTVTQALAAAGVDVPVSCEQGICGTCITRVLEGE |
| | | PDHRDLFLSPEEQARNDQFLPCCSRAKSRVLVLDL* |
| iphD | Acidovorax | MAVMTTLQARIHQLRYEAAGTTSVELRPLPPATQFAQPVEA |
| | wautersii | GAHIDLHLADDLTRSYSLTNPGEAHRYVVAVARDPASRGGS |
| | | RFVHESLRVGQVITIGGPRNHFALDESAPHSVLVAGGIGITPV |
| | | YAMLQRLAVLGRTAHLVYCAGSRAGAAFVADIEALAAASA |
| | | GSITVDWYFKDERGTRADLPRLLAGHPEGTHFYACGPLPLLD |
| | | GYEQACESLGLAHVHLERFAAAPLAPSQTPSQGYDVELRKS |
| | | GKTVHVAPGIALLDALLDAGMNPDYSCREGVCGACEMKVL |
| | | CGEVDHRDLILSKQDQAANRSMMVCVSGCKSGSLVLDF* |
| iphD | Comamonas | MASTYLQARVHQMRYEAAGTLSVELRPLVVAEEFAQPVQA |
| | testosteroni | GAHIDLHLADGLIRSYSLINPGESHRYVVAVSLDPASRGGSRF |
| | | VHQRLRVGDVIQIGGPRNHFPLVETAPHSVLVAGGIGITPVLA |
| | | MLRRLDALGRTAHLIYCASSRASAAFVPEIEAIAAQVGGRVT |
| | | VDWHFKEEKGVRADLHNLLQGHAEGAHFYACGPLAFLDSY |
| | | EDSCGKLGLAHVHLERFAAAPLAAPRTPEVGYAVELRRTGR |
| | | TVQVAAGTSLLDTLINAGMNPEYSCREGVCGACEVRVISGD |
| | | VDHRDQILSEQERAANKSMMICVSGCRSGNLVLDC* |
| MFS | Paraburkholderia | MEKNLASSEGMAAPVWSANDAPAASSADTSPYRWVALFIV |
| Trans- | tuberum | WAAFLLSYVVRVAWSTVAAPVGASLGISVSMLGAFVTAFY |
| porter | | AGYVLANVVGGMLTDLLGGRAMLTLALLPLGVLTFTFGYT |
| | | HSLAFGIVIQAAMGFAAGADYSAGMKIIPAWFTRDRGRAMG |
| | | LYTTATSLAVVIANALVPSFSARHGWSNAFHMLGIVTFGWGI |
| | | VTLLLLRNRPSNEAKPARNSVQEMLGLLRNRNLIALSIAGCG |
| | | GLWATVGFGAWGNALMTRQYGIAPVVAGSIVASFGIGAVIA |
| | | KPTLGWISDLPGVSRRMMSIGCLTAFAIALLVFGYCSTVREF |
| | | YLVAPILGAFSFGYLPVLMAQVSDASGKRLAGASAGWTNAI |
| | | WQSGSAVSPMVVGSLYGASHSFMLALITLAIGPAVAVVAMF |
| | | FVNPHIARE* |

### Example 3—High Atom Economy Conversion of Lignin into Aromatic Monomers Through Oxidative Carbon-Carbon Bond Cleavage

### Introduction

Lignin is the most abundant biobased source of aromatics and a potential renewable feedstock for high value aromatic biochemicals. However, its depolymerization to value-added monomers remains challenging. This is in part due to its complex and heterogeneous structure, comprising an intricate network of C—O and C—C bonds, and its susceptibility to condensation reaction that lead to new C—C bonds.

In the past two decades, several methods have been developed that produce monomers efficiently through cleavage of lignin C—O bonds. Among these, reductive catalytic fractionation (RCF) is highly regarded, as monomer yields through C—O bond cleavage (largely β-O-4) approach the theoretical maximum. While promising, the majority of aromatics in RCF lignin bio-oil are bound within dimers, trimers, and oligomers through recalcitrant C—C linkages, namely β-β, β-5, β-1, and 5-5, rendering them inaccessible by state-of-the-art methods. Similarly, hydrodeoxygenation (HDO) is a reductive process that cleaves C—O bonds and has been widely applied to lignin and lignin models, affording deoxygenated aromatic hydrocarbon compounds for as fuel and lubricant applications. As in RCF oil, the majority of aromatics in HDO oil are bound within C—C linkages.

The presence of C—C bonds in both RCF and HDO oils hampers the possibility of increasing monomer yields from lignin. Hence, over the years, C—C cleavage has received increased attention. To date, only a handful of studies that target C—C cleavage are reported in the literature. Samec and co-workers were able to selectively cleave β-β and β-1 linkages in an RCF oligomer substrate via an oxidative pathway, affording 2,6-dimethoxybenzoquinone in 18 wt % with respect to the mass of the oligomer substrate. Hensen and colleagues recently developed a strategy to cleave simultaneously β-β, β1, β-5, and 5-5 bonds in one single step to yield an oil comprising 54 wt % monocyclic alkanes a 5-fold increase compared to benchmark yields using established C—O cleavage methods. Wang and colleagues developed a one-pot process for the cleavage of C—O and C—C bonds under Ru/NbOPO₄ catalysis, affording arene and cycloalkane monomers in 10-32 wt %, representing a 1.5-fold increase compared to the C—O cleavage benchmark. While these results are promising and demonstrate breakthrough cases of C—C cleavage as a significant contribution to the total monomer yield, the overall yields are not significantly greater than those of state-of-the-art methods that employ C—O cleavage alone. To push the state-of-the-art and achieve higher yields of lignin aromatic monomers, improved methods for C—C bond cleavage are needed.

We recently reported on the autoxidative C—C cleavage of phenol-protected RCF oligomers using two catalytic systems inspired by the Mid-Century process. Notably, these two systems cleave 3 of the 4 types of C—C bond linkages present in RCF oils (β-β, β-5, and β-1) and produce a mixture of monomers with total yields ranging 13-23 wt %, normalized to the mass of the oligomer substrate. Notably, the mixture of products is bioavailable and can be funneled into single products using engineered strains of Pseudomonas putida. Despite its potential, the non-selective nature of autoxidative C—C cleavage necessitates phenol production and RCF monomer separation prior to C—C cleavage, as overoxidation was observed with the oxygenated RCF substrate and led to deleterious degradation of the products.

Herein, we describe the Co/Mn/Br-catalyzed autoxidative C—C cleavage of HDO lignin oil, which affords high yields of bioavailable monomers at 70 wt %. We hypothesized that deoxygenating RCF lignin oil would improve aromatic monomer yields beyond those reported. This was based on preliminary data comparing monomer yields from autoxidative C—C cleavage of propylbenzene (85 mol %) and propylveratrole (13 mol %) under identical conditions. In this example, we assess the effects of various process parameters on monomer yields for process optimization. Using the combination of parameters, we achieved a 70 wt % yield of a mixture comprising primarily benzoic, phthalic, isophthalic, and terephthalic acids, representing a 143 mol % increase compared to monomer content in the HDO oil substrate. Such complex mixture was then biologically funneled into β-muconic lactone, allowing for a high yield of one single compound from lignin.

### Results and Discussion

Substrate preparation by RCF and HDO. We initiated RCF using clean chips of the hybrid poplar clone OP-367-(P. deltoides x P. nigra), which were first refined in a bliss hammermill and passed through a ¼″ sieve prior to reductive catalytic fractionation (RCF). To generate enough material for scale-up, we ran 9 RCF reactions in parallel using a 11.4 L Parr batch reactor vessel. In each run, ca. 300 g of poplar biomass and 15 g of 5% Ru/C were loaded. To maximize delignification, the reactions were run in 2 L methanol with 1 L of water. After pressurizing the vessels with 30 bar H₂ and heating at 225° C. for 3 h, a dark slurry was obtained from which a brown viscous RCF lignin oil could be obtained upon extraction with dichloromethane and removal of the volatiles by rotary evaporation. A total mass of 300.34 g of RCF oil was produced from the 9 reactions.

After RCF and workup, the resulting lignin bio-oil was subjected to hydrodeoxygenation (HDO) over a molybdenum carbide (Mo₂C) catalyst, as has been described previously. This was done in a trickle-bed reactor consisting of a 1.2″ OD reactor tube contained within a single zone split furnace. The oil was flowed at a rate of 0.3 mL/min along with H₂ (270 mL/min STP) over 8.66 g Mo₂C at 350° C. and 900 psi. The resulting product was sampled every 30 min for the duration of the reaction and contained a high concentration of phenolic compounds. Following the reaction, steady state reaction samples were combined, and the formed aqueous phase was aliquoted away. The resulting phenolic mixture was then subjected to a second reaction pass over a fresh Mo₂C bed under intensified process conditions (375° C., 900 psi, 0.3 mL/min, 270 mL/min H₂ STP) to remove any remaining phenolic moieties, resulting in a deoxygenated kerosene rich in aromatic compounds. The resulting carbon yield was measured to be 78 C-mol % with most of the losses likely resulting from demethoxylation.

The HDO oil was subsequently characterized by GC-FID to analyze and quantify the monomer distribution. Using individual analytical standards for quantification of the monomers, we found cycloparaffin and aromatic monomers comprise 45 wt % of the oil, or 3.73 mmol monomers/g of HDO lignin oil. Aromatic monomers make up the majority, encompassing 44 wt % of the oil and totaling 3.43 mmol aromatic monomers/g of HDO lignin oil. Propylbenzene is the largest component of the oil, comprising 2.15 mmol/g (29 wt %), followed by toluene (0.36 mmol/g, 3.3 wt %). The next most abundant aromatic component on a wt % basis is o-methylpropylbenzene (0.20 mmol/g, 2.7 wt %). Ethylbenzene is present in 0.23 mmol/g 2.5 wt %), followed by 0.15 (2.0 wt %) and 0.12 mmol/g (1.6 wt %) of p- and m-methylpropylbenzene, respectively. The remaining aromatic monomers include indane (0.17 mmol/g, 2.0 wt %), o-xylene (0.03 mmol/g, 0.4 wt %), and p-xylene (0.02 mmol/g, 0.2 wt %). We attribute the methyl-substitution on the aromatics to methyl-transfer from the lignin methoxyl groups during HDO, which is commonly observed during HDO of lignin phenolic monomers.

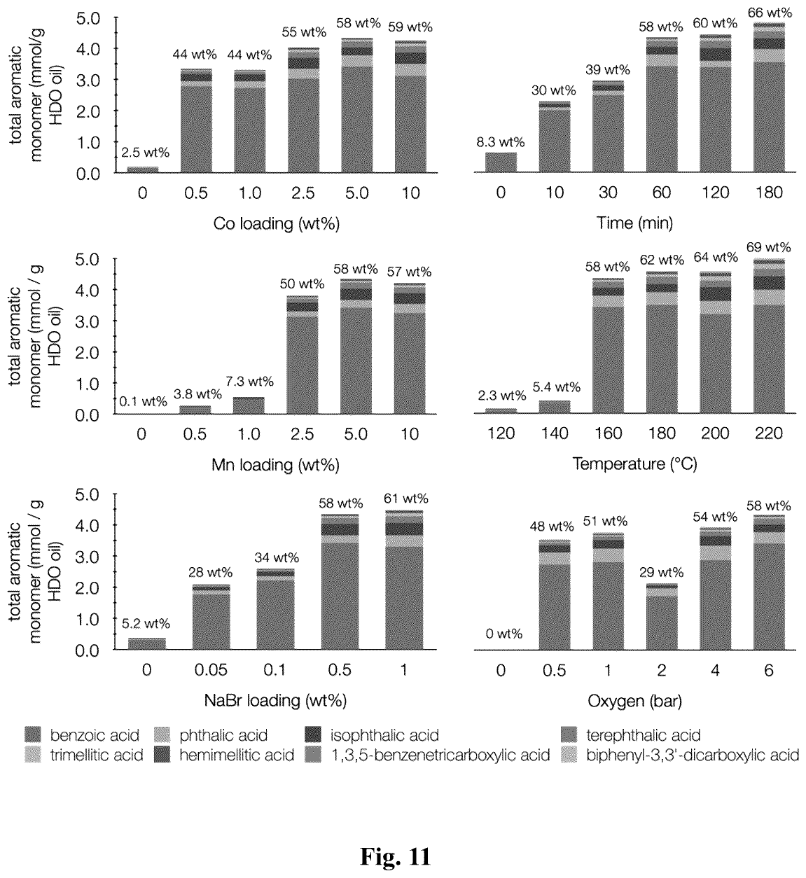

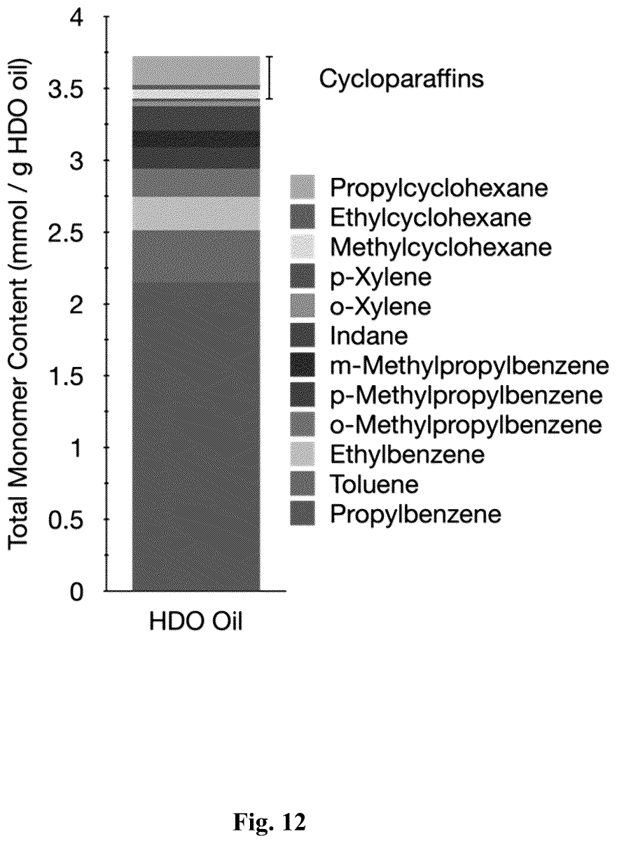

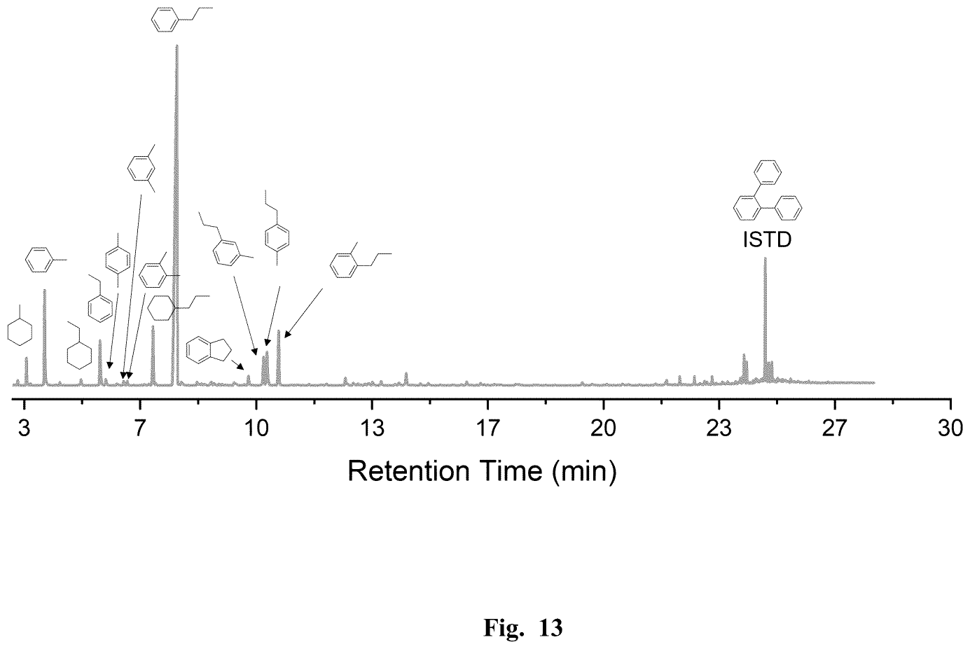

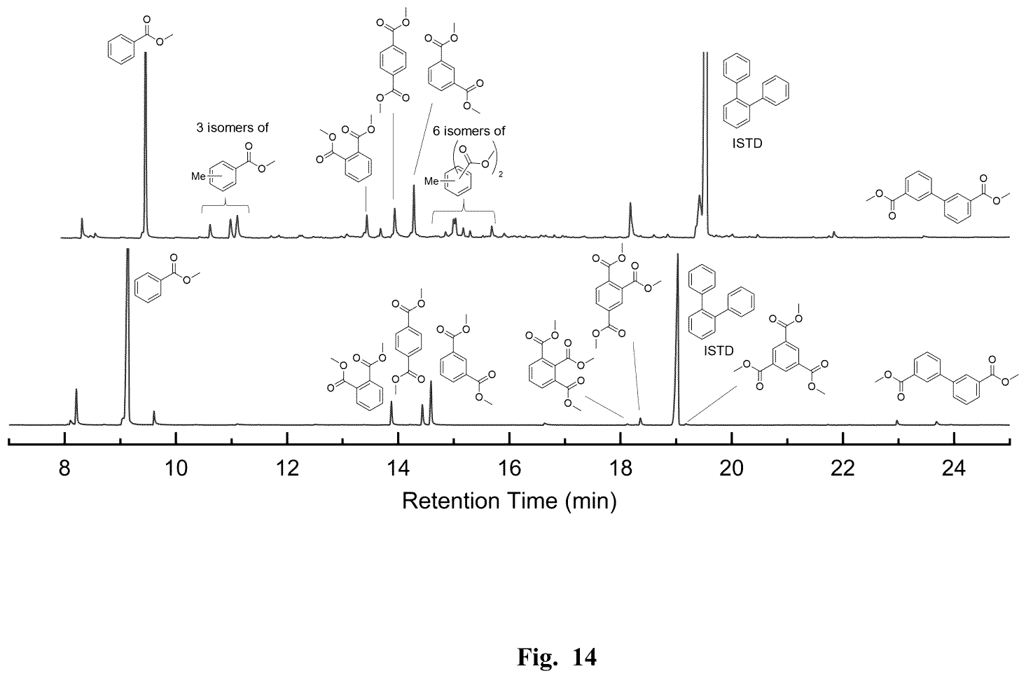

Optimization of the reaction conditions: We devised a set of standard conditions similar to those employed during MC process oxidations. Our optimizations were run in 75 mL Parr titanium batch reaction vessels loaded with 50 mg of HDO oil and 20 mL of acetic acid (AcOH) solvent. Our standard conditions include an operating temperature of 160° C., residence time of 1 h, catalyst loadings of 5/5/0.5 wt % of Co(OAc)₂·4H₂O/Mn(OAc)₂·4H₂O/NaBr, respectively, and an O₂ partial pressure of 6 bar (diluted to 10% in N₂). We maintained these parameters between runs while changing only a single parameter, varied along the x-axis of the stacked column plots in FIG. 11. The detected products were various aromatic mono-, di-, and tri-carboxylic acids: benzoic acid (1), phthalic acid (2), isophthalic acid (3), terephthalic acid (4), trimellitic acid (5), hemimellitic acid (6), trimesic acid (7), biphenyl-3,3′-dicarboxylic acid (8). The presence of other non-characterized products is expected, even if in very low amounts. All of the following optimization data are single runs and will be discussed as wt % with respect to the starting oil.

We initially investigated the effect of temperature on total monomer yields while keeping all other parameters consistent with our standard conditions. FIG. 11 shows data presented in the temperature range 120-220° C. At 120° C., 2.3 wt % of aromatic products were produced. The number increases slightly to 5.4 wt % at 140° C. and substantially to 58 wt % at 160° C. At 180° C. and 200° C., the total aromatic monomer yields were similar, 62 wt % and 64 wt %, respectively. At 220° C., the total aromatic monomer yield increased to 69 wt %. Thus, under our standard conditions, 220° C. demonstrated the highest total monomer yield between these single-run experiments.

Next, we tested whether total monomer yields change when O₂ loadings are varied from 0 to 6 bar, FIG. 11. At 0 bar O₂, we did not detect aromatic monomers, as expected from an autoxidative process. Between 0.5-6 bar O₂, there was a slight but steady increase in total aromatic monomer yields from 48 wt % at 0.5 bar 02 to 56 wt % at 6 bar. We designated 6 bar of 02 partial pressure to be optimal under our standard conditions, as increasing the 02 pressure further approaches the maximum working pressure of our titanium batch reactors.

Maintaining high partial pressures of O₂ is important during Co/Mn/Br-catalyzed autoxidations. If O₂ partial pressures are too low, carbon-centered radicals will couple with one another, leading to condensed structures and cross-linking through C—C bond formation. Despite the 6 bar O₂ pressure used herein is super stoichiometric with respect to the estimated number of cleavable C—C bonds, we continued to see a steady increase in total aromatic monomer yields under our standard conditions. These data demonstrate the importance of mass transport in our batch-reactor configuration to promote alkyl peroxyl formation over C—C bond coupling.

We also altered concentrations of the individual catalyst components, FIG. 11. When any single catalyst component was removed from the reaction, low yields were observed, highlighting the synergy among the catalyst components. For example, a total yield of 2.5 wt % was observed without Co(OAc)₂·4H₂O, 0.1 wt % without Mn(OAc)₂·4H₂O, and 5.2 wt % without NaBr. Adding just 0.5 wt % of Co(OAc)₂·4H₂Oresulted in a sharp increase in total aromatic monomers to 44 wt %. This number further increased to 55 wt % with a 2.5 wt % Co(OAc)₂·4H₂Oloading, and it increased to 58 wt % monomers with 5 wt % Co(OAc)₂·4H₂O. Conversely, increasing Mn(OAc)₂·4H₂Oto 0.5 and 1.0 wt % loadings afforded only modest increases in total monomer yields at 3.8 and 7.3 wt %, respectively. At 2.5 wt % Mn(OAc)₂·4H₂O, a sharp increase to 50 wt % was observed, and loadings of 5.0 and 10 wt % Mn(OAc)₂·4H₂Osaw further increases in yield, ranging 57-58 wt % total aromatic monomers. With NaBr, a steady increase in total monomer yields from 28 wt % at 0.05 wt % NaBr loadings to 58 wt % at 0.5 wt % NaBr. By increasing NaBr loadings to 1 wt % under our standard conditions, only a slight increase to 61 wt % was observed. Ultimately, these catalyst loading studies suggested an optimal catalyst ratio of 5 wt % Co(OAc)₂·4H₂O, 5 wt % Mn(OAc)₂·4H₂O, and 1 wt % NaBr.

Lastly, we tested how total aromatic monomer yields changed with respect to residence time under our standard conditions. At 0 min, i.e. as soon as our reaction vessel reached reaction temperature, we quenched the reaction in an ice bath and quantified a total monomer yield of 8.3 wt %. Similarly, after 10 min, the monomer yield increased to 30 wt %, and after 30 min, 39 wt %. The total aromatic monomer yield continued to increase with prolonged reaction times. At 60 min, a 58 wt % yield was observed, and at 120 min, 60 wt %. Under our standard conditions, the yield continued to increase at 180 min, ultimately affording 66 wt % of monomers.

Hence, with these studies, we determined the optimal conditions to include a Co(OAc)₂·4H₂O/Mn(OAc)₂·4H₂O/NaBr catalyst loading of 5/5/1 wt %, reaction temperature of 220° C., a 3 h residence time, and O₂ loadings of 6 bar.

Reductive catalytic fractionation. The following procedure was performed with poplar (9 runs): Under a flow of nitrogen, 300 g of poplar biomass and 30 g of 5% Ru/C were added to a 7.6 L Parr batch reactor vessel. Next, 1 L of water was added followed by 2 L of methanol. The vessel was pressure tested with dinitrogen (117 bar), where the maximum acceptable loss was 7 mbar/min. Subsequently, the reactor was flushed twice with 27.6 bar of nitrogen to sparge the vessel of any residual air. Once all the nitrogen was drained, the vessel was pressured with hydrogen (30 bar). The mixture was then heated (225° C.) with the mag drive set to 80% of max stirring, and cooling water was run through the mag drive. After 3 h, the vessel was rapidly cooled with water through a cooling coil. The vessel was depressurized, and the product was pumped out of the mixture using a peristaltic pump. The methanol was removed by rotary evaporation, and the residual was extracted with equivolume dichloromethane. The water fraction was extracted two more times with dichloromethane layers were combined, washed with water, dried with sodium sulfate, and ethyl acetate was evaporated by rotary evaporation. A total yield of 300.34 g of lignin oil was obtained.

Trickle-bed Reactor Design/Dimensions: Reactions were performed in a scaled-up trickle bed reactor using a similar design to that reported previously. The reactor consisted of a 21″ long, ½″ OD Hastelloy reactor tube located in a vertically-mounted, insulated, single-zone, split furnace (Applied Test Systems Series 3210) with steel heat transfer blocks to fill void space. Two K-type thermocouples were used for temperature regulation. One thermocouple, contacting the outside of the steel heat transfer blocks was used for temperature control along with a Digi-sense TC9600 PID controller. Meanwhile, the other was slotted to contact the outside of the reactor tube in the center of the heating zone and used to probe reaction temperature. Liquid was fed using a Teledyne ISCO 1000D Syringe Pump, which fed into a ⅛″ OD 316 stainless steel drip tube leading to the heated reaction zone at the top of the reactor. Gas flow rates were controlled using mass flow controllers (Brooks SLA5850S1BAB1C2A1), which fed into the top of the reactor co-currently with the liquid via ½″ 316 stainless steel tubing. Gas/liquid separation was done at room temperature using a Jerguson® glass level gage. Downstream of the gage, a diaphragm back-pressure regulator (Equilibar H3P1SNN8-NSBP1500T100G20KK) was used to maintain system pressure. A separate nitrogen back-fill line was used to maintain constant system pressure during liquid sampling via a needle valve (Swagelok).

The reactors were packed as follows: a quartz wool (Technical Glass Products Inc.) plug was placed in the bottom of the reactor tube, followed by 9.75″ of quartz chips [fused (granular), Sigma-Aldrich, sieved 10-20 mesh], another quartz wool plug, the catalyst bed, another quartz wool plug, and finally more quartz chips up to 1″ below the level of the drip tube. The plugs and quartz packing minimize reactor holdup volume while helping to secure the position of the catalyst bed.

HDO Catalyst Synthesis: Catalyst synthesis was done as previously reported. Briefly, ammonium molybdate tetrahydrate (AMT) (ACS reagent grade, Fisher Scientific) was sieved between 60 and 100 mesh. Next, 15 g of AMT was then loaded into the reactor to generate 8.6 g of Mo₂C. A heating ramp from 25° C. to 680° C. was done over 3.5 h, then held for another 3.5 h under 165 mL/min hydrogen (UHP, Airgas) and 45 mL/min of methane (UHP, Airgas). Methane flow was then stopped and a subsequent 30-minute scavenging step was done under pure hydrogen gas to clear the catalyst surface of polymeric carbon. The reactor was then cooled under hydrogen flow prior to the reaction start.

Feed Preparation: Neat lignin oil was used after generation via RCF using the procedure outlined above. Approximately 8-10 batches of oil were combined, resulting in 300-400 grams of feed in total. Recycled lignin oil was prepared as described previously. The steady-state first-pass reaction samples were combined after removing enough sample for analysis. The water fraction was allowed to phase-separate from the combined sample and removed via pipette.

Autoxidation: For a single reaction, a Parr grade 4 corrosion resistant titanium batch reactor fit with a glass liner insert was charged with 50 mg of HDO oil, acetic acid (20 mL), a stir bar, and catalyst components Co(OAc)₂·4H₂O, Mn(OAc)₂·4H₂O, and NaBr. The mixture was pressurized three times with an inert gas and subsequently charged with 10% air to achieve the desired partial pressure of oxygen. The vessel was heated to 200° C., at which temperature it was held for 3 h before it was cooled rapidly in an ice bath. The solutions were stored in the freezer until needed for analysis.

HPLC Quantification: Monomer quantification of 1,2,4 benzenetricarboxylic acid, hemimellitic acid, 1,3,5 benzenetricarboxylic acid, biphenyl-3,3′ dicarboxylic acid, phthalic acid, isophthalic acid, terephthalic acid (TPA), and benzoic acid was performed using the chromatographic conditions described. Briefly, analysis performed using an Infinity 111290 ultra-high-performance liquid chromatography (UHPLC) system (Agilent Technologies) equipped with a G7117A diode array detector and coupled with a Zorbax Eclipse Plus C18 Rapid Resolution HD (2.1×50 mm, 1.8 μm) column. A gradient of 20 mM phosphoric acid and methanol was used to achieve separation of desired analytes. The calibration was evaluated from 1-1000 ug/mL for 1,2,4 benzenetricarboxylic acid, hemimellitic acid, 1,3,5 benzenetricarboxylic acid, biphenyl-3,3′ dicarboxylic acid, and benzoic acid. The other calibration standards (TPA, phthalic acid, and isophthalic acid) were evaluated from 1-250 ug/mL. All compounds were analyzed at a wavelength of 240 nm.

### Example 4—Genetically Modified P. Putida for the Conversion of Benzoic Acid, Terephthalate, Isophthalate and Ortopthalate to Muconolactone

Pseudomonas putida KT2440 has the following genetic modifications which enable to conversion of benzoic acid (BA), terephthalate (TPA), isophthalate (IPA) and ortho-phthalate (OPA) to muconolactone: ΔhsdMR::P_{tac}:tphA2_{II}:tphA3_{II}:tphB_{II}:tphA1_{II}fpva:P_{tac}:tpaK ΔaldB::P_{tac}:iphE_{Paraburkholderia tuberumTM52}:P_{tac}:iphA_{TM55}:iphD_{TM55}:iphB_{Comamonas sp. E6}ΔampC::P_{tac}:ophC_{AG8816}:ophK_{AG8816}Δcrc::P_{tac}:OphA2_{Comamonas sp. E6}:OphA1_{Comamonas sp. E6}:ophB_{Comamonas sp. E6}:ophP_{Comamonas sp. E6}ΔpcaD.

fpva:P_{tac}:tpaK_{RHA1} enables terephthalate uptake and tpaK is a heterologously expressed gene from Rhodococcus jostii RHA1.

ΔhsdMR::P_{tac}:tphA2II:tphA3II:tphBII:tphA1II_{E6} enables terephthalate conversion to protocatechuate and tphA2II, tphA3II, tphBII, and tphA1II are heterologously expressed genes from Comamonas sp. E6.

ΔaldB::P_{tac}:iphE_{Paraburkholderia tuberumTM52}:P_{tac}:iphA_{TM55}:iphD_{TM55}:iphB_{Comamonas sp. E6} enables isophthalate uptake (iphE) and conversion to protocatechuate. iphE_{Paraburkholderia tuberumTM52} is a heterologously expressed gene from Paraburkholderia tuberum TM52. iphATM55 and iphDTM55 are heterologously expressed genes from an Acidovorax wautserii environmental isolate, TM55. iphB is a heterologously expressed gene from Comamonas sp. E6.

ΔampC::P_{tac}:ophC:ophK Δcrc::P_{tac}:ophA2:ophA1:ophB:ophP enables ortho-phthalate uptake and conversion to protocatechuate. ophT is an outer membrane porin and ophK is an inner membrane MFS transporter. ophA1, ophA2, ophB, and ophT are heterologously expressed genes from Comamonas sp. E6. ophC and ophK are heterologously expressed genes from a Pseudomonas sp. SXM-1environmental isolate, AG8816.

The deletion of pcaD (ΔpcaD) and prevents catabolism of muconolactone, enabling the build-up of muconolactone when benzoic acid, terephthalate, isophthalate and ortho-phthalate are fed to the engineered strain.

We initiated construction of our strain for the conversion of the primary aromatic monomers (benzoic acid (BA), terephthalic acid (TPA), isophthalic acid (IPA) and ortho-phthalic acid (OPA)) from a oxidized HDO lignin (HDOx) to a single exemplary product by introducing the catabolic pathways for isophthalic acid (IPA) and ortho-phthalic acid (OPA) into the terephthalic acid-consuming strain: TDM461 (P. putida KT2440 ΔhsdMR::P_{tac}:tphA2_{II}:tphA3_{II}:tphB_{II}:tphA1_{II} fpva:P_{tac}:tpaK). Fortunately, wildtype Pseudomonas putida KT2440 utilizes benzoic acid, so no further engineering efforts were required for BA consumption. Catabolic pathways for IPA and OPA were optimized by Austin Carroll at ORNL; plasmids for the homologous integration of the IPA and OPA catabolic pathways were shared with NREL for strain construction. Successful integration of the IPA and OPA catabolic pathways into TDM461 resulted in the strain AW521: P. putida KT2440 ΔhsdMR::P_{tac}:tphA2_{II}:tphA3_{II}:tphB_{II}:tphA1_{II} fpva:P_{tac}:tpaK ΔaldB::P_{tac}:iphE_{Paraburkholderia tuberumTM52}:P_{tac}:iphA_{TM55}:iphD_{TM55}:iphB_{Comamonas sp. E6} ΔampC::P_{tac}:ophC:P_{tac}:OPA transporter Δcrc::P_{tac}:ophA2:ophA1:ophB:P_{JE151211}:OPA porin.

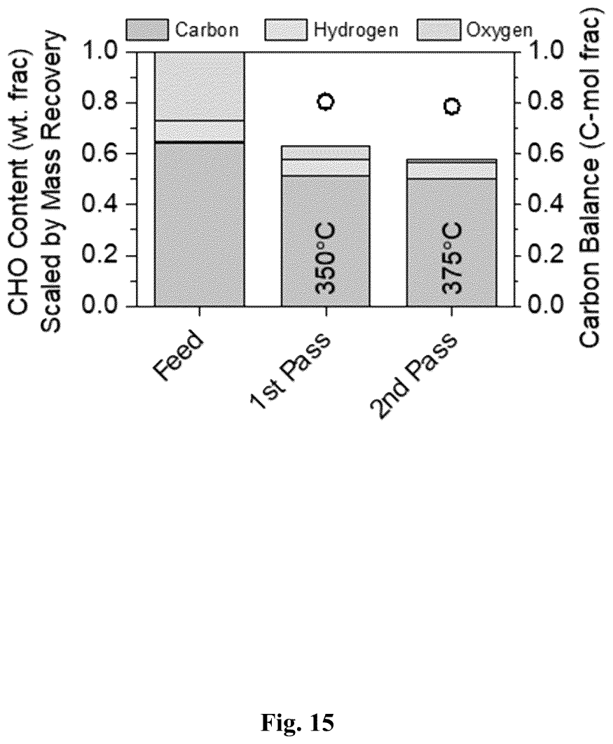

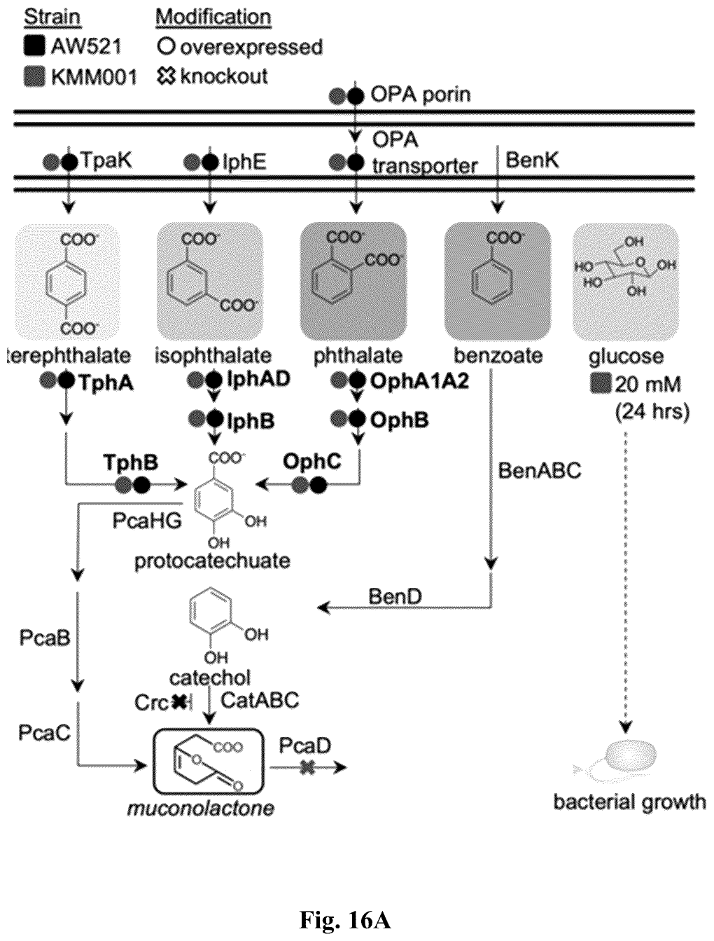

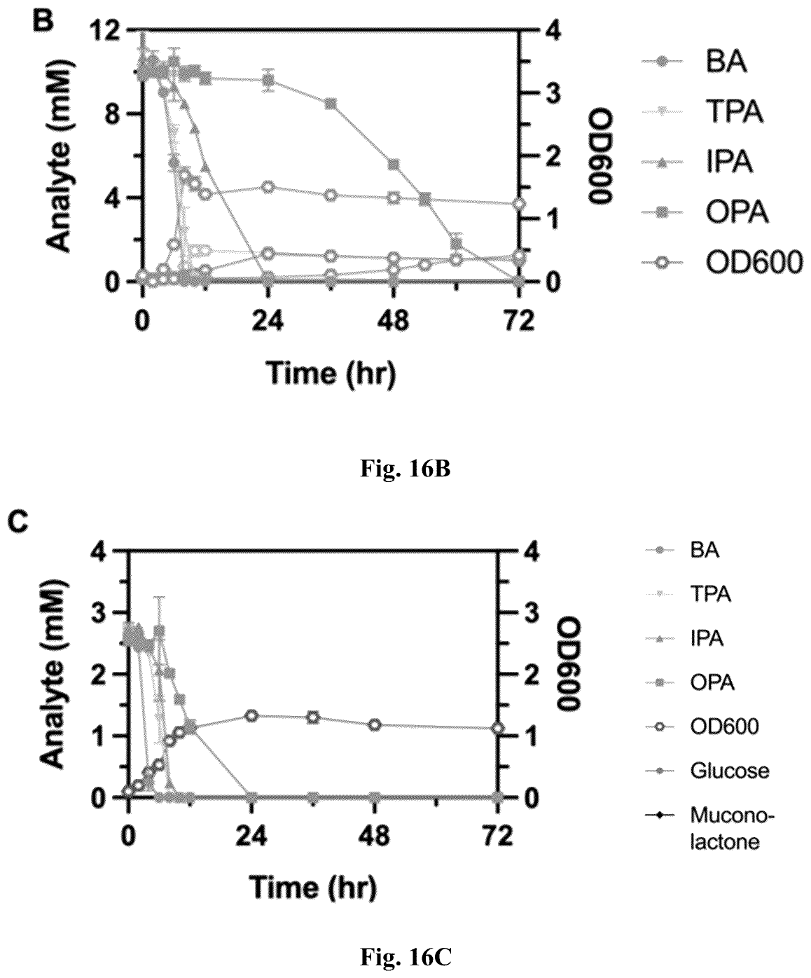

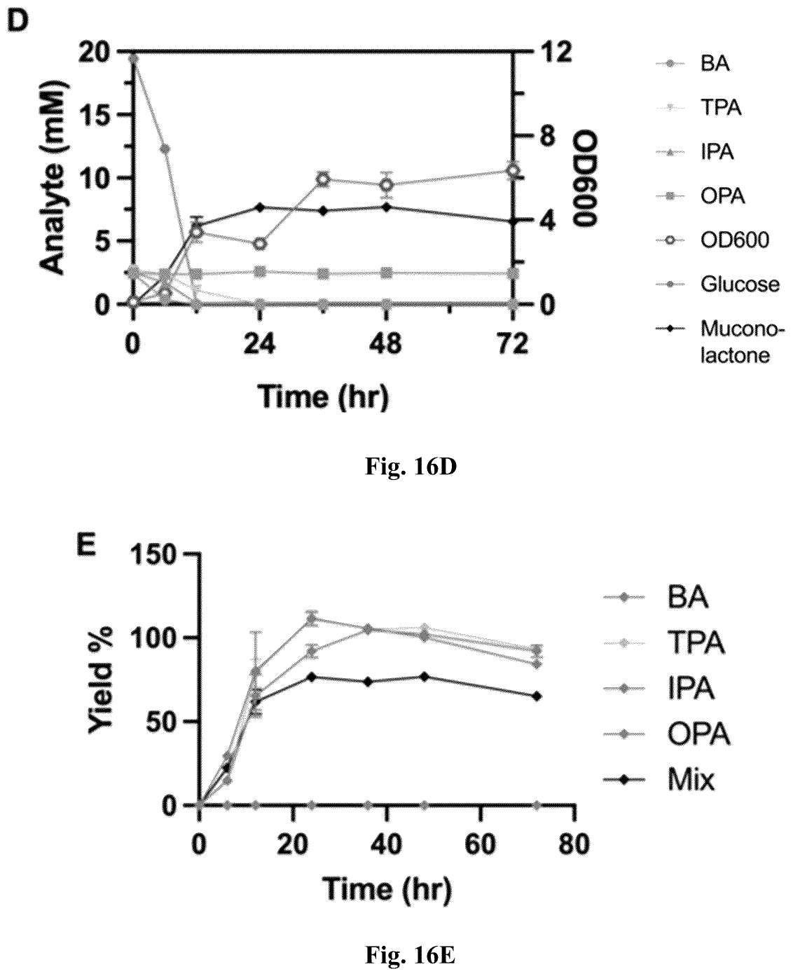

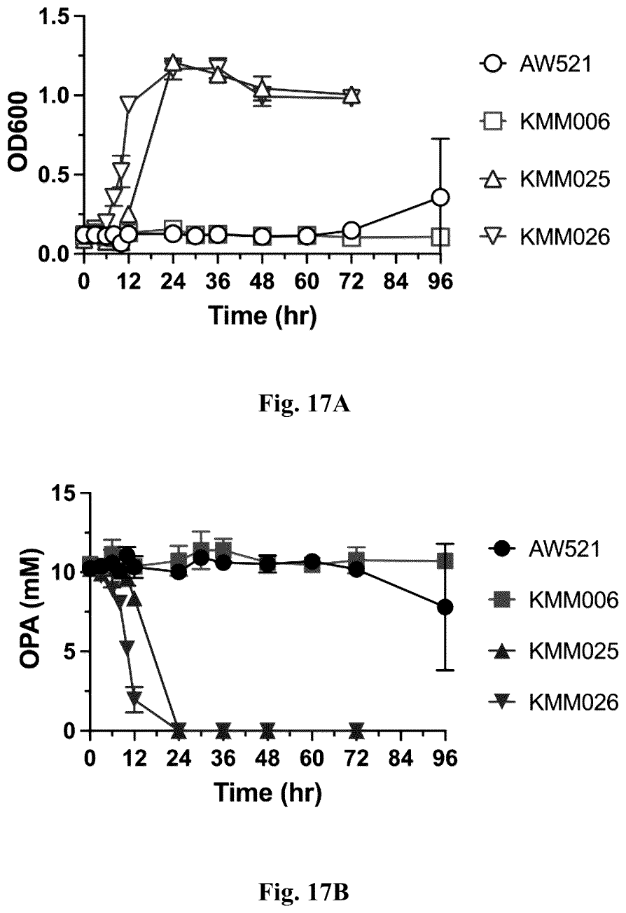

After successful construction of AW521, we confirmed the ability of AW521 to grow on all four HDOx monomers as the sole carbon source (10 mM) or in a mix case (10 mM total aromatics, 2.5 mM of each monomer) in a shake flask study (FIGS. 15B-15C). The strain consumed all BA, TPA and IPA within 24 hours post inoculation, but did not begin consuming OPA until around 24 hours and did not utilize all OPA until 72 hours post inoculation when individual monomers were the sole carbon source (FIG. 15C). When all four monomers were present as carbon sources (i.e., the “mix” case; FIG. 15C), AW521 consumed all monomers, including OPA, within 24 hours. From this experiment, we determined the utilization of all four HDOx monomers by AW521 was satisfactory enough to move forward with knocking out pcaD to enable muconolactone production.

We deleted the gene pcaD, which converts muconolactone to β-ketoadipic acid, in AW521 to generate the strain KMM001. This deletion should enable the accumulation of muconolactone from HDOx monomers when KMM001 is fed glucose for biomass accumulation. To confirm the conversion of HDOx monomers to muconolactone by KMM001, we performed a shake flask study with 10 mM of each HDOx monomer individually or 2.5 mM each HDOx monomer in a mix (10 mM aromatics total); we fed 20 mM glucose every 24 hours to enable growth. KMM001 successfully converted BA, TPA, and IPA to muconolactone at near 100% yields in both the individual monomer cases and in the mix case (FIGS. 15D-15E). However, the strain did not utilize OPA in either the individual monomer case or in the mix case (FIGS. 15D-15E).

We generated an engineered strain, AW521, that utilizes all four major HDOx monomers (BA, TPA, IPA, and OPA) for growth. We selected muconolactone as our single exemplary target and deleted pcaD from AW521, generating strain KMM001, to enable muconolactone accumulation from HDOx monomers. In a shake flask study, we showed KMM001 successfully converts BA, TPA, and IPA to muconolactone at high yields. However, poor OPA utilization rates by AW521 resulted in the inability of KMM001 to convert OPA to muconolactone. Therefore, we performed an adaptive laboratory evolution study in which AW521 was grown serially on 10 mM OPA. We identified mutations in the ribosome binding site of the OPA MFS transporter in the adapted strains and reverse engineered AW521 to include two of these mutations, generating strains KMM025 and KMM026. While both KMM025 and KMM026 had improved growth on OPA compared to AW521, KMM026 showed the fastest growth. Therefore pcaD will be knocked out of KMM026 to enable muconolactone production from BA, TPA, IPA, and OPA.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

When a group of substituents is disclosed herein, it is understood that all individual members of that group and all subgroups, are disclosed separately. When a Markush group or other grouping is used herein, all individual members of the group and all combinations and subcombinations possible of the group are intended to be individually included in the disclosure. For example, when a device is set forth disclosing a range of materials, device components, and/or device configurations, the description is intended to include specific reference of each combination and/or variation corresponding to the disclosed range.

Every formulation or combination of components described or exemplified herein can be used to practice the invention, unless otherwise stated.

Whenever a range is given in the specification, for example, a density range, a number range, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter is claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A method comprising:
providing a lignocellulosic biomass reactant;
fractionating the lignocellulosic biomass reactant via reductive catalytic fractionation (RCF), thereby generating a RCF oil;
deoxygenating the RCF oil via hydrodeoxygenation (HDO), thereby generating a HDO oil;
oxidizing the HDO oil, thereby generating a plurality of oxygenated monomers; and
bioconverting the plurality of oxygenated monomers in the presence of a genetically engineered Psuedomonas putida bacterium, thereby generating muconic acid.

2. The method of claim 1, wherein the lignocellulosic biomass reactant comprises lignin.

3. The method of claim 1, wherein the step of fractionating is performed in the presence of an RCF catalyst.

4. The method of claim 4, wherein the RCF catalyst comprises Mo₂C.

5. The method of claim 1, wherein the step of fractionating has a greater than 90% conversion of the lignocellulosic biomass reactant to RCF oil.

6. The method of claim 1, wherein the step of deoxygenating is performed in the presence of a of an HDO catalyst.

7. The method of claim 6, wherein the HDO catalyst comprises Mo₂C.

8. The method of claim 1, wherein the step of deoxygenating has a greater than 90% conversion of the RCF oil to HDO oil.

9. The method of claim 1, wherein said step of oxidizing is performed in the presence of an oxidation catalyst.

10. The method of claim 9, wherein the oxidation catalyst comprises one or more of Co(OAc)₂, Mn(OAc)₂, Zr acetylacetonate and NaBr.

11. The method of claim 9, wherein the oxidation catalyst comprises Co(OAc)₂, Mn(OAc)₂ and NaBr at a ratio of 5:5:1, respectively.

12. The method of claim 9, wherein each oxidation catalyst is provided at a weight percentage selected from the range of 0.5 to 10 wt %.

13. The method of claim 1, wherein the step of oxygenating has a greater than 80% conversion of HDO oil to oxygenated monomers.

14. The method of claim 1, wherein the oxygenated monomers comprise benzoic acid, phthalic acid, terephthalic acid, isophthalic acid, hemimellitic acid, benzene tricarboxylic acid, biphenyl dicarboxylic acid or a combination thereof.

15. A genetically modified bacterium comprising:
a genetically modified strain of Psuedomonas putida KT2440, wherein the Psuedomonas putida KT2440 is capable of converting benzoate and terephthalate into muconate.

16. The bacterium of claim 16, having the modification fpva:P_{tac}:tpaKRHA1 where fpva:P_{tac}:tpaKRHA1 enables terephthalate uptake and tpaK is a heterologously expressed gene from Rhodococcus jostii RHA1.

17. The bacterium of claim 16, having the modification ΔhsdMR::Ptac:tphA2II:
tphA3II:tphBII:tphA1IIE6, where ΔhsdMR::Ptac:tphA2II:tphA3II:tphBII:tphA1IIE6 enables terephthalate conversion to protocatechuate and tphA2II, tphA3II, tphBII, and tphA1II are heterologously expressed genes from Comamonas sp. E6.

18. The bacterium of claim 16 having the modification ΔcatRBC::Ptac:catA, where ΔcatRBC::Ptac:catA enables catechol conversion to muconate and prevents catabolism of muconate.

19. The bacterium of claim 16 having the modification ΔampC::P_{tac}:ophC:ophK Δcrc::P_{tac}:ophA2:ophA1:ophB:ophP enabling ortho-phthalate uptake and conversion to protocatechuate.

20. The bacterium of claim 16, wherein pcaD (ΔpcaD) has been deleted to prevent the catabolism of muconolactone.
